(19) United States
(12) Patent Application Publication
(10) Pub. No.: US 2024/0415741 A1
(43) Pub. Date: Dec. 19, 2024

(54) SYSTEMS, APPARATUS, AND METHODS FOR PLACING A GUIDEWIRE FOR A JEJUNOSTOMY TUBE

(71) Applicant: CoapTech, Inc., Baltimore, MD (US)

(72) Inventors: Steven P. TROPELLO, Baltimore, MD (US); William Alexander LeMay PATTERSON, Dundalk, MD (US); Jared WINEBERG, Baltimore, MD (US); Rachel SWANSTROM, Blaine, MN (US)

(73) Assignee: CoapTech, Inc., Baltimore, MD (US)

(21) Appl. No.: 18/442,867

(22) Filed: Feb. 15, 2024

Related U.S. Application Data
(63) Continuation of application No. PCT/US2022/040941, filed on Aug. 19, 2022.
(60) Provisional application No. 63/236,630, filed on Aug. 24, 2021.
(60) Provisional application No. 63/235,028, filed on Aug. 19, 2021.

(51) Int. Cl. A61J 15/00; A61B 8/08; A61B 17/00; A61B 17/34; A61M 5/00; A61M 39/10

(52) U.S. Cl. CPC A61J 15/0069; A61B 8/0841; A61B 17/00234; A61B 17/3415; A61J 15/0007; A61J 15/0049; A61M 5/007; A61M 39/105; A61B 2017/00358; A61B 2017/00818; A61M 2205/0288

(57) ABSTRACT

Devices, systems, and methods for guidewire placement for a jejunostomy tube are described herein. A system can include an elongated tube having a first end, a second end, and an engagement portion at the first end configured for engagement with an endoscope. An inflatable member can be coupled to the elongated tube and configured to transition from an uninflated configuration to an inflated configuration. A magnetic member can be coupled to the elongated tube and configured such that, in response to disposing an external magnetic assembly on a surface of the abdomen of the patient, the inflatable member can be urged against an inner surface of a jejunum wall of the patient such that any intervening structure between the surface of the abdomen of the patient and the inner surface of the jejunum wall is disposed between the external magnetic assembly and the inflatable member with substantially no fluid gaps.

### CROSS-REFERENCE TO RELATED APPLICATIONS

This application is a continuation of International patent application No. PCT/US2022/040941, filed Aug. 19, 2022, entitled “Systems, Apparatus, and Methods for Placing a Guidewire for a Jejunostomy Tube,” which claims priority to and the benefit of U.S. Provisional Application No. 63/235,028, filed Aug. 19, 2021, entitled “Systems, Apparatus, and Methods for Placing a Guidewire for a Jejunostomy Tube,” and U.S. Provisional Application No. 63/236,630, filed Aug. 24, 2021, entitled “Systems, Apparatus, and Methods for Placing a Guidewire for a Jejunostomy Tube,” the entire contents of each of which are incorporated by reference herein for all purposes.

This application is related to U.S. patent application Ser. No. 17/080,441, filed Oct. 26, 2020, now U.S. Pat. No. 11,612,546, entitled “Systems, Apparatus, and Methods for Placing a Guidewire for a Gastrostomy Tube,” the entire contents of which is hereby expressly incorporated by reference for all purposes.

### BACKGROUND

Embodiments described herein relate to systems, apparatus, and methods for placing a guidewire for a jejunostomy tube to provide access to a patient's jejunum via a route bypassing the upper gastrointestinal (GI) tract of the patient.

Percutaneous jejunostomy is a common approach to deliver post-pyloric enteral nutrition directly into the small intestinal lumen, bypassing the mouth, esophagus, and stomach, for patients who cannot tolerate oral or gastric feeding. Patients suffering from many medical conditions may require jejunostomy tube (J-tube) placement, including various cancers, gastroparesis and other digestive disorders, and high-risk for aspiration (common for many hospitalized patients). See Panagiotakis P H, DiSario J A, Hilden K, Ogara M, Fang J C. DPEJ tube placement prevents aspiration pneumonia in high-risk patients. Nutr Clin Pract 2008 April-May; 23(2):172-5. doi: 10.1177/0884533608314537. PubMed PMID: 18390785; Kim CY, Engstrom BI, Horvath JJ, Lungren MP, Suhocki PV, Smith TP. Comparison of primary jejunostomy tubes versus gastrojejunostomy tubes for percutaneous enteral nutrition. J Vasc Interv Radiol 2013 December; 24(12):1845-52. doi: 10.1016/j.jvir.2013.08.012. Epub 2013 Oct. 1. PubMed PMID: 24094674, the disclosures of which are incorporated herein by reference. ‘J-tube’ placement procedures are increasingly performed, and account for over 40,000 procedures annually in the US. Despite its common indication, jejunostomy remains a high-risk and technically challenging procedure presently: it requires puncturing through the abdominal wall and safely advancing a needle directly through a crowded anatomical region with several other vital organs, followed by hitting a piece of free-floating, hard-to-target small bowel, before safely securing a feeding tube for long-term use.

Thus, jejunostomy is performed by highly skilled specialists, namely gastroenterologists using endoscopy, interventional radiologists (IR) using fluoroscopy, and surgeons using laparoscopy. Gastroenterologists commonly perform Direct Percutaneous Endoscopic Jejunostomy (DPEJ) by feeding an endoscope down past the mouth, esophagus, and stomach, whereby a safe tract into the jejunum is attempted to be identified by transillumination. Once a needle puncture is made into the jejunum, a guidewire is introduced through the needle and snared under endoscopic visualization. The guidewire is drawn all the way back out of the mouth. Finally, a jejunostomy tube is fed via the wire through the mouth and advanced back down into the jejunum. Alternatively, the radiologic method (percutaneous radiological jejunostomy, or PRJ) uses fluoroscopic guidance in which IR physicians extend a nasoenteric tube over a guidewire into the jejunum. The jejunal loop is identified on fluoroscopy by air insufflation and/or contrast to guide needle puncture. The jejunum is then mechanically pexied to the abdominal wall to secure it in place before a larger tract can be formed for external J-tube insertion.

Conventional percutaneous jejunostomy procedures are inherently complicated and technically challenging. A significant difficulty is associated with the jejunum being a small free-floating organ that is constantly peristalsing. Jejunal loops can lie deep within the peritoneum, may be obstructed by other vital anatomy, and can be difficult to needle target due to movement. See Gray R R, Ho C S, Yee A, et al. Direct percutaneous jejunostomy. AJR Am J Roentgenol 1987; 149:931e2; Van Overhagen H, Ludviksson MA, Lameris J S, et al. US and fluoroscopic-guided percutaneous jejunostomy: experience in 49 patients. J Vasc Interv Radiol 2000; 11:101e6, the disclosures of which are incorporated herein by reference. Reliably maintaining the jejunal loop positioning while avoiding puncturing interposed anatomy is therefore extremely difficult. Along with an inability to affix the jejunum before needle entry, neither endoscopes nor fluoroscopy can fully “see through” the local tissue and thus proceduralists are making an educated guess as to the needle's path to the target jejunum. One study of DPEJ procedures indicated that in up to 10% of cases at least one transabdominal needle pass did not enter the visualized jejunal lumen, but rather went blind into the surrounding region.

These technical limitations lead to higher rates of adverse events and procedural failures. J-tube malposition by mistakenly puncturing through interposed anatomy commonly occurs. DPEJ complications vary from 4.2%-23%, including higher rates of enterocutaneous fistula. Such complications may require advanced surgical drainage, IV antibiotics, and surgical intervention for correction while being associated with higher mortality rates. DPEJ aborts occur in as many as 30% of cases and naturally are directly correlated with patient size and BMI. Ability to transilluminate is a key variable, with only ˜60% procedural success witnessed when tract depths exceed 3 cm. A summary of procedural risks is presented in Table 1.

TABLE 1
Summary of Percutaneous Jejunostomy Risks

| Procedure | Risk / Limitation |
|---|---|
| DPEJ/PRJ | “Blind Stick” leading to common occurrence of |
| | enterocutaneous fistula |
| DPEJ/PRJ | Free floating jejunum leading to erroneous needle sticks |
| DPEJ | Low success rates in obese population |

Thus, there is a need for systems, apparatus, and methods of placing a jejunostomy tube which reduce risks to the patient and allow for the jejunostomy tube to be quickly and easily placed and secured in communication with the jejunum.

### SUMMARY

Systems, apparatus, and methods for placing a guidewire for a jejunostomy tube are described herein. In some embodiments, a system includes an elongated tube having a first end and a second end, the elongated tube including an engagement portion on the first end of the elongated tube configured for engagement with an endoscope such that the endoscope can translate the first end of the elongated tube. Further the system includes an inflatable member coupled to the elongated tube, the inflatable member configured to transition from an uninflated configuration to an inflated configuration. Also the system includes a magnetic member coupled to the elongated tube, the magnetic member configured such that, in response to disposing an external magnetic assembly on a surface of the abdomen of the patient, the inflatable member can be urged against an inner surface of a jejunum wall of the patient to urge the jejunum wall toward the surface of the abdomen such that any intervening structure between the surface of the abdomen of the patient and the inner surface of the jejunum wall is disposed between the external magnetic assembly and the inflatable member with substantially no fluid (e.g., liquid or gas, such as air) gaps.

In some embodiments, a method includes translating a first end of an elongated tube through an orifice of a patient, through an esophagus and/or stomach of the patient, and into a jejunum of the patient such that an inflatable member and a magnetic member of the elongated tube are disposed in the jejunum of the patient. An external magnetic assembly can be disposed on a surface of the patient such that the magnetic member of the elongated tube is urged toward the surface of the patient and the inflatable member is disposed against an inner surface of a jejunum wall of the jejunum. The inflatable member can be inflated via a lumen of the elongated tube such that the inflatable member transitions from an uninflated configuration to an inflated configuration.

### BRIEF DESCRIPTION OF THE DRAWINGS

FIG. 1 is a schematic illustration of a portion of a patient, according to an embodiment.

FIG. 2 is a schematic illustration of a guidewire placement system, according to an embodiment.

FIG. 3 is a flow chart of a method, according to an embodiment.

FIG. 4 is a schematic illustration of a guidewire extended through the esophagus, stomach, and jejunum of a patient, according to an embodiment.

FIG. 5 is a schematic illustration of an inflation assembly of a system extended through the esophagus, stomach, and jejunum of a patient, according to an embodiment, in an uninflated configuration.

FIG. 6 is a schematic illustration of the inflation assembly of FIG. 5 with the inflation assembly in an uninflated configuration and an external magnetic assembly of the system of FIG. 5.

FIG. 7 is a schematic illustration of the inflation assembly of FIG. 5 with an inflatable member of the inflation assembly in an inflated configuration, according to an embodiment.

FIG. 8 is a schematic illustration of the inflation assembly of FIG. 5 adjacent to an ultrasound probe of the system of FIG. 5, according to an embodiment.

FIG. 9 is a schematic illustration of the system of FIG. 5 including a needle penetrating an inflatable member of the inflation assembly, according to an embodiment.

FIG. 10 is a schematic illustration of the system of FIG. 5 with a guidewire assembly extending through the needle into the inflatable member.

FIG. 11 is a schematic illustration of the system of FIG. 5 with the needle removed.

FIG. 12 is a schematic illustration of the system of FIG. 5 with the guidewire assembly partially pulled into the stomach and/or within a proximal portion of a proximal bowel by the inflation assembly.

FIG. 13 is a schematic illustration of the system of FIG. 5 with the guidewire assembly extending from the outside of the patient, through the orifice, through the esophagus, through the stomach, through the jejunum, and outside the patient via an access opening.

FIG. 14 is a schematic illustration of the system of FIG. 5 with the guidewire assembly separated from the inflation assembly.

FIG. 15 is a schematic illustration of the system of FIG. 5 with a coupling member separated from the guidewire assembly.

FIG. 16 is a schematic illustration of the system of FIG. 5 with a feeding tube threaded over the guidewire assembly.

FIG. 17 is a schematic illustration of the system of FIG. 5 with the feeding tube engaged with the jejunum wall and extending through the access opening in the jejunum wall.

FIG. 18 is schematic illustration of a T-fastener for fastening a wall of jejunum to an abdominal wall, according to an embodiment.

FIG. 19 is a schematic illustration of the system of FIG. 5 including a needle penetrating a jejunum in proximity of the inflatable member of the inflation assembly, according to an embodiment.

FIGS. 20-23 are schematic illustrations of a method of placing a needle through a wall of jejunum, according to an embodiment.

FIGS. 24A and 24B are ultrasound images depicting a needle penetrating a jejunum, according to an embodiment.

FIGS. 25-27 are various views of an inflatable assembly, according to an embodiment.

FIGS. 28-29 are perspective views of a portion of an inflatable assembly and an engagement portion of the inflatable assembly, respectively, according to an embodiment.

FIGS. 30-31 are images of a jejunostomy procedure using an inflatable assembly having an engagement portion and an endoscope, according to an embodiment.

FIGS. 32A and 32B are schematic illustrations of a an engagement portion of an inflatable assembly and an endoscope and a flexible loop, respectively, according to various embodiments.

FIGS. 33-36 are various views of a portion of an inflatable assembly and an endoscope having a coupling mechanism, according to an embodiment.

FIGS. 37-38 are various views of a portion of an inflatable assembly and an endoscope having a snare mechanism, according to an embodiment.

FIGS. 39-41 are various views of an inflation assembly, according to an embodiment.

FIGS. 42-44 are various views of the inflation assembly of FIGS. 39-41 with an endoscope disposed therein, according to an embodiment.

### DETAILED DESCRIPTION

As described above, jejunostomy procedures are complicated due to a jejunum being a free-floating organ that is difficult to access from an outside of an abdominal wall for placing a jejunostomy tube (or for any other suitable procedure). Thus, systems, apparatus, and methods are described herein for facilitating access to a jejunum from the outside of the patient. Specifically, in some embodiments, systems, apparatus, and methods are described herein for locating a particular region of a jejunum that needs to be accessed from the outside of a patient and fixing the region of the jejunum in place relative to an outer surface of a patient (e.g., a surface of the patient's abdomen) by urging the region of the jejunum against an abdominal wall of the patient (or patient's organs in proximity to an abdominal wall of the patient). In some embodiments, such urging of the jejunum against the abdominal wall of the patient (or other suitable organs of the patient) can be achieved with an inflatable member (e.g., a balloon) coupled to an elongated tube and inserted into the jejunum via translation of the elongated tube (or any other suitable mechanism for delivering the inflatable member into jejunum). It should be noted that, while description of the present invention is directed towards jejunostomy procedures, in some embodiments, similar systems and procedures may be conducted for any other suitable cavities of a human body that may require access from an outside of the patient. For example, similar systems and methods may be used for accessing a stomach of a patient, or any other suitable cavity, vessel, and the like.

In some embodiments, a system includes an elongated tube, an inflatable member, and a magnetic member. The elongated tube includes a first end and a second end. Further, the elongated tube includes an engagement portion on the first end of the elongated tube configured for engagement with an endoscope such that the endoscope can translate the first end of the elongated tube. The inflatable member can be coupled to the elongated tube and is configured to transition from an uninflated configuration to an inflated configuration. The magnetic member can be coupled to the elongated tube, and is configured such that, in response to disposing an external magnetic assembly on a surface of the abdomen of the patient, the inflatable member can be urged against an inner surface of a jejunum wall of the patient to urge the jejunum wall toward the surface of the abdomen such that any intervening structure between the surface of the abdomen of the patient and the inner surface of the jejunum wall is disposed between the external magnetic assembly and the inflatable member with substantially no fluid gaps.

In some embodiments, a method can include translating an inflatable member of an elongated tube through an orifice of a patient, through an esophagus and/or stomach of the patient, and into a jejunum of the patient, such that the inflatable member and a magnetic member of the elongated tube are disposed in the jejunum of the patient. Translating the inflatable member can include translating a first end of the elongated tube through the orifice of a patient, through the esophagus and the stomach of the patient, and into the jejunum of the patient. An external magnetic assembly can be disposed on a surface of the patient such that the magnetic member of the elongated tube is urged toward the surface of the patient and the inflatable member is disposed against an inner surface of a jejunum wall of the jejunum. The inflatable member can be inflated via a lumen of the elongated tube such that the inflatable member transitions from an uninflated configuration to an inflated configuration.

In some embodiments, a system includes an elongated tube, an inflatable member, and a magnetic member. The elongated tube includes a first end and a second end. Further, the elongated tube defines a lumen configured to receive an endoscope such that the endoscope can translate the first end of the elongated tube. The inflatable member can be coupled to the elongated tube and is configured to transition from an uninflated configuration to an inflated configuration. The magnetic member can be coupled to the elongated tube, and is configured such that, in response to disposing an external magnetic assembly on a surface of the abdomen of the patient, the inflatable member can be urged against an inner surface of a jejunum wall of the patient to urge the jejunum wall toward the surface of the abdomen such that any intervening structure between the surface of the abdomen of the patient and the inner surface of the jejunum wall is disposed between the external magnetic assembly and the inflatable member with substantially no fluid gaps.

In some embodiments, a kit includes an inflation assembly and a guidewire assembly. The inflation assembly includes an elongated tube, an inflatable member, and a magnetic member. The elongated tube includes a first end and a second end. The elongated tube is configured for engagement with an endoscope such that the endoscope can translate the first end of the elongated tube. The inflatable member can be coupled to the elongated tube and is configured to transition from an uninflated configuration to an inflated configuration. The magnetic member can be coupled to the elongated tube, and is configured such that, in response to disposing an external magnetic assembly on a surface of the abdomen of the patient, the inflatable member can be urged against an inner surface of a jejunum wall of the patient to urge the jejunum wall toward the surface of the abdomen such that any intervening structure between the surface of the abdomen of the patient and the inner surface of the jejunum wall is disposed between the external magnetic assembly and the inflatable member with substantially no fluid gaps. The guidewire assembly includes a guidewire and a coupling member. The guidewire assembly can be configured to be translated through an opening in an abdomen of the patient such that the coupling member engages with the inflatable member in the jejunum and the guidewire extends through the abdomen of the patient.

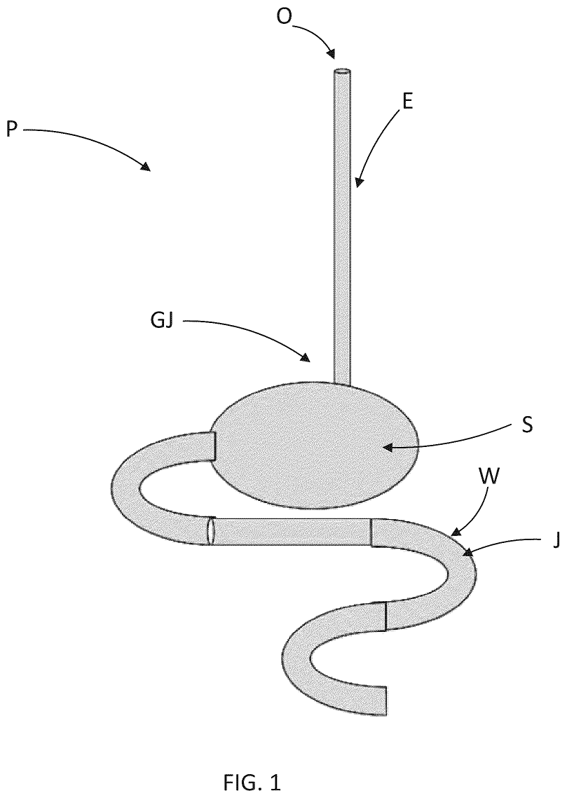

FIG. 1 is a schematic illustration of a portion of a patient P. The patient P has an orifice O, an esophagus E, a stomach S, and a jejunum J. The orifice O can be, for example, an oral orifice or a nasal orifice. The orifice O is coupled to the esophagus E, the esophagus E is coupled to the stomach S at the gastroesophageal junction GJ, and the stomach is coupled to the jejunum J. Thus, the jejunum J is accessible from the orifice O via the esophagus E and stomach S. The jejunum J includes a jejunum wall W such that the interior of the jejunum J can be accessed via piercing the jejunum wall W.

When the patient P has difficulty swallowing food and/or liquid, a jejunostomy tube (also referred to herein as a “J-tube”) can be placed via a jejunostomy tract created in the jejunum wall W such that nutrition can be provided directly through the jejunostomy tube to the jejunum. For example, the jejunum wall W can be serially dilated to create the gastrostomy tract and insert a J-tube. As another example of J-tube placement, a first end of a stiff catheter can be fluoroscopically directed through a preformed passage in the jejunum wall W of jejunum, through the stomach S, and through the esophagus E to the gastroesophageal junction J. After the first end of the stiff catheter has been translated through the gastroesophageal junction J, a guidewire can be translated through the catheter, out of the first end of the catheter, and through the orifice O of the patient. A J-tube can then be translated over the guidewire and secured in the jejunostomy tract in engagement with the jejunum wall W. For example, the J-tube can be pushed over the guidewire from the orifice O, through the esophagus E, into the stomach S, and into engagement with the jejunum wall W. Alternatively, the J-tube can be coupled to the first end of the guidewire (e.g., a looped end) extending from the orifice O and pulled through the orifice O, through the esophagus E, into the stomach S, and into engagement with the jejunum wall W by the guidewire. Navigation of the catheter and the guidewire through the patient, however, can be challenging and pose risks to the patient. For example, the extended use of fluoroscopy to navigate the stiff catheter through the patient carries the risk of radiation-induced injuries to the patient. Furthermore, if internal guidance (e.g., fluoroscopy) is not used to navigate the catheter and guidewire through the patient, the catheter may need to be stiffer to traverse the route through the patient. Catheters with increased stiffness, however, are more likely to damage tissue via unintended tears and/or perforations. Furthermore, the jejunum may have a tendency to move within the abdomen and can be difficult to secure relative to an opening or tract through a patient's skin (e.g., through a patient's abdomen).

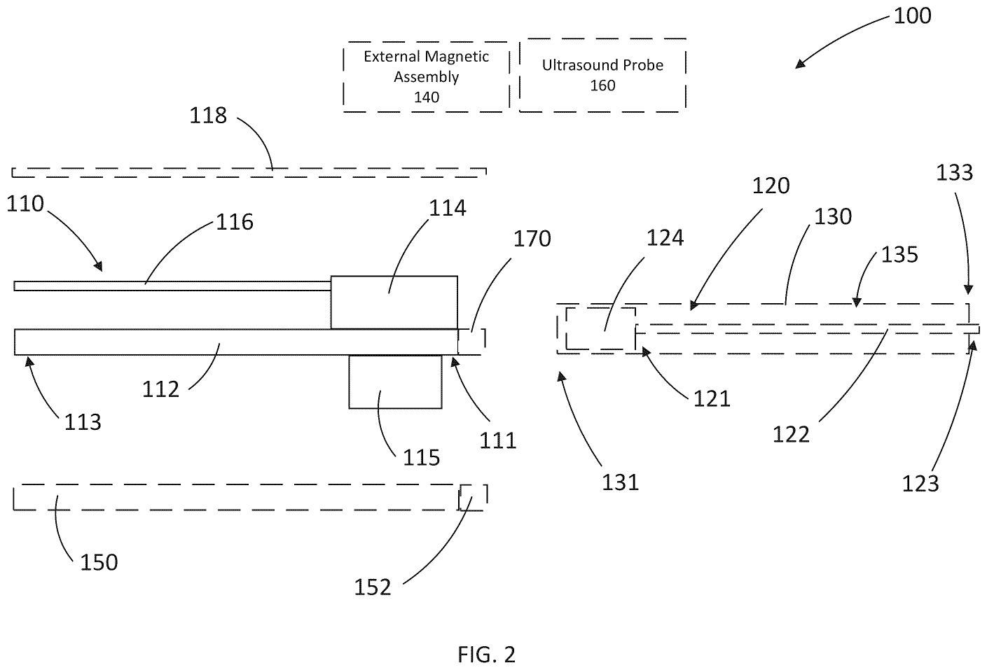

In some embodiments, however, access to the jejunum can be created via a patient's abdomen and a jejunum wall of the patient. For example, FIG. 2 is a schematic representation of a system 100. The system 100 includes an elongated tube, an inflation assembly 110, and a magnetic member 115. The inflation assembly 110 can include an elongated tube 112 and an inflatable member 114. The elongated tube 112 can have a first end 111 and a second end 113. In some embodiments, the elongated tube 112 can have a length sufficient to extend from at least an oral or nasal orifice of a patient to the jejunum of a patient via an esophagus and a stomach of the patient. The inflatable member 114 can be coupled to the elongated tube 112 at or near the first end 111 of the elongated tube 112. The inflation assembly 110 can include an inflation lumen 116 in fluid communication with the inflatable member 114. In some embodiments, the inflation lumen 116 can be disposed within and/or be defined by the elongated tube 112.

As shown in FIG. 2, the elongated tube 112 can optionally include an engagement portion 170. The engagement portion 170 can be disposed on the first end 111 of the elongated tube 112. The engagement portion 170 can include any suitable device or mechanism or be formed to have any suitable shape for engaging with an endoscope (e.g., endoscope 150) described in more detail below) to couple elongated tube 112 with the endoscope such that the endoscope can control movement (e.g., translational movement) of the elongated tube 112. In some embodiments, for example, engagement portion 170 can include a tip portion of the elongated tube 112 and can define an opening. In some embodiments, a flexible loop member (also referred to herein as a flexible loop) can be threaded through the opening such that a portion of the flexible loop is disposed within the opening. Thus, the flexible loop threaded through the opening can be engaged (e.g., grasped) to control movement of the engagement portion 170 and the elongated tube 112. In some embodiments, the tip portion can define a circumferential groove about a perimeter of the top portion. The circumferential groove can be shaped to receive a snare (e.g., a flexible loop) of an endoscope such that the endoscope can control movement of the engagement portion 170 and the elongated tube 112. In some embodiments, rather than a flexible loop, the system 100 can include a rigid, substantially rigid, or partially rigid loop used similarly.

As shown in FIG. 2, the system 100 can optionally include or be configured to be used in conjunction with a guidewire 118 and/or an endoscope 150. The endoscope 150 can include an endoscope coupling mechanism 152. The endoscope coupling mechanism 152 can be used to couple the endoscope 150 with the engagement portion 170. For example, in some embodiments, the endoscope coupling mechanism 152 can include or be formed as a flexible loop (e.g., a snare) configured to engage with the elongated tube 112 (e.g., with a circumferential groove of the elongated tube 112). In some embodiments, the endoscope coupling mechanism 152 can include or be formed as mechanical graspers (e.g., hinged jaws) manipulatable to grasp a portion of the engagement portion 170 of the elongated tube 112 (e.g., a flexible loop threaded through an opening defined by the engagement portion 170) and to pull the flexible loop to advance, retract, and/or steer the elongated tube 112. In some embodiments, the elongated tube 112 can define an endoscope lumen or channel within which the endoscope 150 can be disposed such that navigation (e.g., steering) of the endoscope 150 through the patient carries an end of the elongated tube 112 to a target location.

In some embodiments, the inflatable member 114 can surround the elongated tube 112 in an inflated and/or uninflated configuration. In some embodiments, the inflatable member 114 can extend laterally from the elongated tube 112 in an inflated and/or uninflated configuration. In some embodiments, the inflatable member 114 can extend distally from the first end 111 of the elongated tube 112 in an inflated and/or uninflated configuration. In some embodiments, the inflatable member 114 can be disposed on the elongated tube 112 such that a portion of the elongated tube 112 extends distally of the inflatable member 114 when the inflatable member 114 is in an inflated and/or uninflated configuration. In some embodiments, the inflatable member 114 can have two ends (e.g., cuffs), and each end can be sealed to an outer surface of the elongated tube 112. The elongated tube 112 can define one or more inflation holes such that the inflation lumen 116 can be in fluid communication with the interior of the inflatable member 114 for transitioning the inflatable member 114 between an uninflated and an inflated configuration. In some embodiments, the inflatable member 114 can be formed on or as a part of a rigid subassembly, and the rigid subassembly can receive the elongated tube 112 within an orifice of the subassembly and the elongated tube 112 can then be sealed to the subassembly.

In some embodiments, the inflatable member 114 can be formed in any suitable shape, in any suitable size, and of any suitable material. For example, the inflatable member 114 can be elliptical, spherical, cylindrical, rectangular, tear drop, or any other suitable shape. In some embodiments, the shape can be chosen based on the particular application of the system 100. For example, the shape of the inflatable member 114 may be selected to improve ultrasound visualization in particular regions of a patient's body. Furthermore, the inflatable member 114 can be sized for improved engagement and retention between the inflatable member 114 and the guidewire assembly 120.

The inflatable member 114 can be sufficiently pliable such that the inflatable member 114 (e.g., when inflated) can be punctured (e.g., by a needle) to define a pinhole in the wall of the inflatable member 114 rather than bursting or tearing as a result of puncture. In some embodiments, the inflatable member 114 can be formed of, for example, polyurethane, silicone, and/or polyvinyl chloride (PVC). In some embodiments, the inflatable member 114 can have any suitable material properties, wall thicknesses, and/or inflated outermost diameters.

In some embodiments, for example, the inflatable member 114 can be elliptical in shape and formed of a low durometer urethane. The inflatable member 114 can have an outermost diameter ranging from about 40 mm to about 55 mm in an inflated configuration, and a length of about 55 mm. The inflatable member 114 can have a diameter at each end ranging from about 5.46 mm to about 5.72 mm. The wall thickness at the maximum balloon diameter in the inflated configuration can be between about 0.029 mm and about 0.038 mm. The inflatable member 114 can be filled with up to, for example, about 50 ml of fluid in the inflated configuration. In some embodiments, the inflatable member 114 may have a volume of about 10 ml, about 20 ml, about 30 ml, about 40 ml, and the like in the inflated configuration. In some embodiments, the inflatable member 114 may have a volume of between about 10 ml and about 50 ml, between about 20 ml and about 50 ml, between about 30 ml and about 50 ml, between about 40 ml and about 50 ml, between about 20 ml and about 40 ml, between about 30 ml and about 40 ml, between about 10 ml and about 20 ml, between about 10 ml and about 30 ml, between about 10 ml and about 40 ml, and/or between about 20 ml and about 30 ml. In some embodiments, the inflatable member 114 may be configured to be partially inflated for coaptation.

In some embodiments, the system 100 may optionally include or be used in conjunction with an ultrasound probe 160 configured for visualization of the inflatable member 114 within the patient and any intervening patient structure between the skin of the patient and the inflatable member 114. The ultrasound probe 160 can be any suitable ultrasound probe configured for visualization of the inflatable member 114 within the patient and any intervening patient structure between the skin of the patient and the inflatable member 114. For example, the ultrasound probe 160 may be used to visualize any intervening patient tissue or patient structures such as a wall of the jejunum, and/or any tissues or structures disposed between the jejunum and an abdominal wall. In some embodiments, the system 100 can be used with respect to other body cavities (e.g., a trachea) and other tissues can be visualized using the ultrasound probe 160, such as a cartilage (e.g., a thyroid cartilage), blood vessels such as arteries and/or veins, nerves, and/or any other structures or tissues that may be disposed between the inflatable member 114 and the skin of the patient.

Additionally, or alternatively, the system 100 can include or be used in conjunction with an external magnetic assembly 140. The external magnetic assembly 140 can be any suitable external magnetic assembly configured to urge the magnetic member 115 of the inflation assembly 110 (e.g., via magnetic attraction) toward the external magnetic assembly through patient tissue (e.g., through the skin and jejunum wall of the patient). The ultrasound probe 160 can be used to identify a location of the inflatable member 114 and the magnetic member 115 within the patient P relative to other tissue or structures of the patient P. For example, when the inflatable member 114 is disposed within a cavity (e.g., a jejunum) of the patient, the external magnetic assembly 140 can be coupled to an external surface of the patient (e.g., the anterior surface of the skin of the patient's abdomen) such that the magnetic member 115 of the inflation assembly 110 is urged toward the external magnetic assembly 140 and the inflatable member 114 contacts a surface of a wall of the cavity (e.g., a surface of a wall of the jejunum). The magnetic interaction (e.g., attraction) between the magnetic member 115 and the external magnetic assembly 140 can cause the inflatable member 114 to urge at least a portion of the jejunum (e.g., the portion the inflatable member 114 contacts) towards the external magnetic assembly 140, and thus toward the external surface of the patient. The surface of the wall of the cavity and the external surface of the patient may be disposed on opposite sides of at least one tissue surface of the patient. In some embodiments, the urging can result in the inflatable member 114 compressing tissue and/or structures disposed between the inflatable member 114 and the external surface of the patient. Thus, the inflatable member 114 and the external magnetic assembly 140 can be disposed on opposite sides of any intervening tissue and/or structures such that substantially no fluid (e.g., air) gaps are disposed between the inflatable member 114 and the external magnetic assembly 140. The inflatable member 114 can be visualized within the cavity (e.g., with the inflatable member 114 urging the cavity wall toward the external magnetic assembly 140). For example, the inflatable member 114 can be echogenic and visualized via the ultrasound probe 160. The ultrasound probe 160 can be disposed sufficiently near or adjacent the external magnetic assembly 140 such that the inflatable member 114 urges the cavity wall toward the ultrasound probe 160. This technique, in which an echogenic member is urged against a surface of a wall of a body cavity, and the echogenic member and all tissue planes between the echogenic member and the external surface of the patient can be visualized by ultrasound, can be referred to as Coaptive Ultrasound (CU).

In some embodiments, the external magnetic assembly 140 can include a handle. In some embodiments, the external magnetic assembly 140 can include one magnetic element configured for magnetic interaction with the magnetic member 115. In some embodiments, the external magnetic assembly 140 can include any suitable number of magnetic elements (e.g., two magnetic elements) configured for magnetic interaction with the magnetic member 115. In some embodiments, as described above, the inflation assembly 110 can include a number of magnetic members 115 (e.g., two magnetic members), and the external magnetic assembly 140 can include a corresponding number of magnetic elements.

In some embodiments, the external magnetic assembly 140 and/or the magnetic member 115 can be formed of any suitable type of magnet. For example, the external magnetic assembly 140 and/or the magnetic member 115 can include a permanent magnet, such as a neodymium iron boron (NdFeB) magnet, a samarium cobalt (SmCo) magnet, an aluminum nickel cobalt (AlNiCo) magnet, a ceramic magnet, a ferrite magnet, and/or any other suitable rare earth magnet. In some embodiments, the external magnetic assembly 140 and/or the magnetic member 115 can include a temporary magnet. In some embodiments, the external magnetic assembly 140 and/or the magnetic member 115 can be an electromagnet, such as a solenoid. In some embodiments, the external magnetic assembly 140 and/or the magnetic member 115 can generate a magnetic field having an orientation (i.e., north (N) and south(S) poles). In other embodiments, the external magnetic assembly 140 and/or the magnetic member 115 can be formed of a ferromagnetic material that is not magnetized, i.e. does not generate its own magnetic field, but can be affected by an externally-applied magnetic field. For example, the external magnetic assembly 140 and/or the magnetic member 115 can be formed of iron or steel, and application of an external magnetic field can attract the iron toward the source of the field, applying a force to the external magnetic assembly 140 and/or the magnetic member 115.

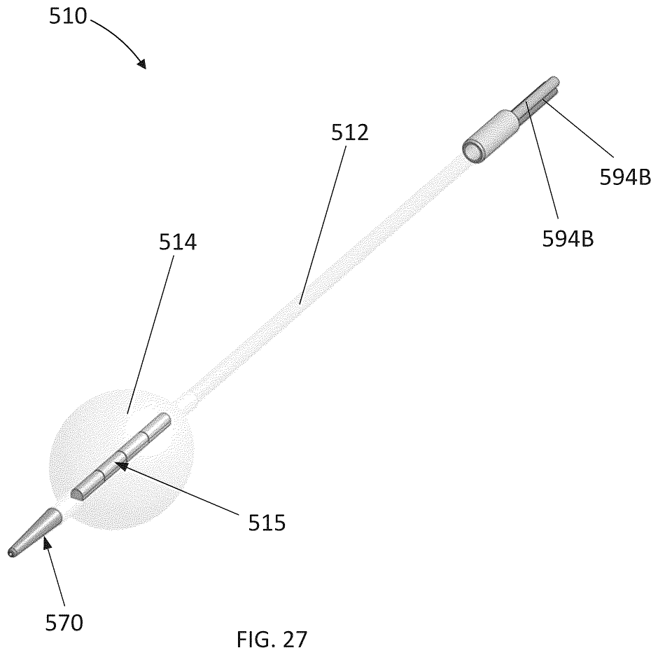

In some embodiments, the magnetic member 115 includes a plurality of discrete magnetic segments configured to move independently from adjacent magnetic segments. The discrete magnetic segments can be disposed (e.g., serially) within a lumen of the elongated tube 112. The elongated tube 112 can be flexible and the discrete magnetic segments can be moved independent relative to one another such that the portion of the elongated tube 112 to which the discrete magnetic segments are coupled is able to flex and bend as the elongated tube 112 is translated through tortuous anatomy (e.g., the jejunum). In some embodiments, the plurality of discrete magnetic segments can be coupled to (e.g., disposed within a lumen of) the elongated tube 112 at an end portion of elongated tube 112. In some embodiments, all or some of the plurality of discrete magnetic segments can be disposed within the inflatable member 114. In some embodiments, all or some of the plurality of discrete magnetic segments can be disposed distally or proximally of the inflatable member 114. In some embodiments, each magnetic segment of the plurality of discrete magnetic segments can be formed as a magnetic sphere, a magnetic block (e.g., an elongated rectangular or square block), a magnetic cylinder, and/or any other suitable shape. In some embodiments, one or more of the discrete magnetic segments can be spaced from an adjacent discrete magnetic segment. In some embodiments, one or more of the discrete magnetic segments can be disposed in contact with an adjacent discrete magnetic segment (e.g., regardless of whether the elongated tube 112 is in a configuration having one or more curves (e.g., a bent configuration) or a straight configuration). In some embodiments, the magnetic segments can each be solid and non-flexible, but can be arranged as described herein such that the magnetic member 115 is flexible. In some embodiments, the discrete magnetic segments can be suspended in a liquid disposed within the lumen of the elongated tube 112 (e.g., formed as magnetic balls suspended in a fluid). In some embodiments, a magnetic fluid may be disposed within a portion of the elongated tube 112 and/or the inflatable member 114. In some embodiments, the magnetic member 115 can have any of the features or characteristics of any of the magnetic members described herein, such as magnetic member 515 described with respect to FIG. 27.

The system 100 can optionally include a guidewire assembly 120. The guidewire assembly 120 can include a guidewire 122 having a first end 121 and a second end 123 and a coupling member 124 disposed at the first end 121 of the guidewire 122. The coupling member 124 can be configured to couple to the inflatable member 114 such that, when coupled, translation of the inflation assembly 110 (e.g., translation of the elongated tube 112 via pulling on the second end 113) can translate the guidewire assembly 120. For example, if the inflatable member 114 is moved in a first direction due to a force applied to the elongated tube 112, the coupling of the coupling member 124 to the inflatable member 114 can cause the coupling member 124 and the guidewire 122 to also move in the first direction. The coupling member 124 can be configured to couple with the inflatable member 114 via, for example, being captured by the inflatable member 114, caught within an interior region of the inflatable member, or engaged with a surface of the inflatable member 114.

In some embodiments, the inflation assembly 110, the guidewire assembly 120, and/or the coupling member 124 can be the same or similar in structure and/or function to any of the inflation assemblies, the guidewire assemblies, and the coupling members, respectively, described in U.S. patent application Ser. No. 17/080,441 to Tropello et al., entitled “Systems, Apparatus, and Methods for Placing a Guidewire for a Gastrostomy Tube” (hereinafter the '441 Application) which was incorporated by reference above for all purposes. For example, in some embodiments, the coupling member 124 can be distinct from the guidewire 122 and fixedly coupled to the guidewire 122 (e.g., via adhesive). For example, in some embodiments, the coupling member 124 can include a first magnetic member configured to couple to a second magnetic member of the inflatable member 114. In some embodiments, the guidewire 122 can include the coupling member 124. For example, the coupling member 124 can be monolithically formed with a shaft of the guidewire 122 such that the guidewire assembly 120 is a one piece structure. Similarly, in some embodiments, the coupling member 124 and the guidewire 122 can be formed of the same material or materials. In some embodiments, the coupling member 124 can be shaped such that the coupling member 124 can engage with at least one portion of a wall of the inflatable member 114. For example, the coupling member 124 can have a planar or a multi-planar shape and can be formed as a pigtail, hook, coil, or corkscrew-shaped end to the guidewire 122. Thus, in some embodiments, the first end 121 of the guidewire 122 can be retained within or near the inflatable member 122 by the coupling member 124 when the coupling member 124 is disposed within the inflatable member 114. In some embodiments, the coupling member 124 can be disposed outside of the inflatable member 114 with the guidewire 122 passing through a first wall portion and a second, oppositely disposed wall portion of the inflatable member 114 such that the guidewire 122 is retained by the inflatable member 114 due to the interaction between the coupling member 124 and the first wall portion of the inflatable member 114. In some embodiments, the coupling member 124 can be partially disposed within the inflatable member 114 and partially disposed outside of the inflatable member 114 such that the guidewire assembly 120 is coupled to the inflatable member 114 for translation of the guidewire assembly 120 via movement of the inflation assembly 110.

In some embodiments, the coupling member 124 can be configured to transition between a first configuration for insertion and a second configuration for retention or coupling. For example, the coupling member 124 can have a smaller lateral extent (e.g., outermost diameter) relative to a central axis of the guidewire 122 in the first configuration than in the second configuration such that the coupling member 124 can fit inside the lumen 135 of the needle 130 in the first configuration and can expand to retain the guidewire 124 relative to the inflatable member 114 in the second configuration. In some embodiments, the coupling member 124 can have a first shape in the first configuration and a second shape in the second configuration such that the coupling member 124 can travel through an opening in at least one sidewall of the inflatable member 114 in the first configuration and can engage a sidewall of the inflatable member 114 in the second configuration such that the coupling member 124 is retained by the inflatable member 114. In some embodiments, the coupling member 124 can be biased toward the second configuration such that, in the absence of external forces on the coupling member 124, the coupling member 124 will assume the second configuration. In some embodiments, in the first configuration the coupling member 124 can be elongated such that the coupling member is shaped as a straight wire. The second configuration can correspond to an unbiased shape or configuration of the coupling member (e.g., a pigtail, hook, coil, or corkscrew-shape). In some embodiments, the guidewire 122 and/or the coupling member 124 can be formed of a shape-memory material such as, for example, Nitinol.

In some embodiments, when the coupling member 124 is within the lumen 135 of the needle 130, the needle 130 can compress the coupling member 124 such that the coupling member is in the first configuration. Thus, the coupling member 124 can have a smaller lateral extent relative to a central axis of the guidewire 124 (e.g., outermost diameter) when disposed within the lumen 135 of the needle 130) than when not within the needle 130. In some embodiments, the lumen 135 and the coupling member 124 can be structured and sized such that the coupling member 124 can be straight or substantially straight within the lumen 135 of the needle 130. For example, the lumen 135 can have an inner diameter similar to an outer diameter of the coupling member 124 (e.g., an outer diameter of a wire forming the coupling member 124 portion of the guidewire assembly 120) such that the coupling member 124 can be laterally compressed to a shape with a smaller outer diameter and/or elongated within the lumen 135 of the needle 130. In some embodiments, the outer diameter of a wire forming the coupling member 124 and the inner diameter of the lumen 135 can be relatively sized such that the outer diameter of the wire forming the coupling member 124 is slightly smaller than the inner diameter of the lumen 135 and the coupling member 124 and the inner surface of the needle 130 defining the lumen 130 can have a slip fit engagement. Thus, when the coupling member 124 is threaded into the lumen 135 of the needle 130, the wire forming the coupling member 124 is straightened out to correspond to the shape of the lumen 135. As the coupling member 124 is translated out of the first end 131 of the needle 130, the coupling member 124 can transition from the first configuration to the second configuration. For example, as the coupling member 124 is extended from the first end 131 of the needle 130, the portion of the coupling member 124 extending from the first end 131 can transition toward the second configuration due to being biased toward the second configuration, while the portion of the coupling member 124 remaining within the lumen 135 of the needle 130 can remain in the first configuration. When the coupling member 124 is entirely outside of the needle 130, the coupling member 124 can be entirely in the second configuration.

In some embodiments, the coupling member 124 can be configured to be translated in a first direction by the inflatable member 114 if a translation force on the inflatable member 114 is greater than a force in a direction opposite of the translation force on the coupling member 124. If the force on the coupling member 124 is opposite and greater than the translation force on the inflatable member 114, the coupling member 124 and the inflatable member 114 can be configured to decouple. For example, in some embodiments in which the coupling member 124 is a pigtail-shaped end to the guidewire 122, the application of sufficient force to the coupling member 124 in a direction opposite a force being applied to the inflatable member 114 can cause the pigtail-shaped end to straighten and decouple from the inflatable member 114. Thus, in some embodiments the coupling member 124 and the inflatable member 114 can be decoupled via applying oppositely directing pulling forces to each of the coupling member 124 and the inflatable member 114. In some embodiments, the coupling member 124 and the inflatable member 114 can be engaged such that the release force (e.g., via oppositely directing pulling forces) necessary to separate the coupling member 124 from the inflatable member 114 is a force greater than the maximum force applied to the guidewire 122 (and therefore coupling member 124) in an opposite direction than the inflatable member 114 during withdrawal of the coupling member 124 from the patient via pulling on the inflation assembly 110. Thus, the release force is sufficiently high such that the inflatable member 114 and the coupling member 124 will not be separated during the withdrawal of the coupling member 124 of the guidewire 122 inadvertently during withdrawal, but can be separated via, for example, pulling by the user when the inflatable member 114 and the coupling member 124 are outside of the patient's body. For example, in some embodiments, the release force can be at least about 0.25 lbs of force, at least about 0.5 lbs of force, or at least about 1.5 lbs of force. In some applications of the system 100, such as non-jejunostomy applications, the release force may be greater or smaller depending on the resistive forces the coupling member 124 and guidewire 122 may experience during withdrawal via a withdrawal force on the inflation assembly 110.

In some embodiments, the coupling member 124 can be configured to pierce the inflatable member 114 such that the coupling member 124 can be inserted into and/or through the inflatable member 114. In some embodiments, the system 100 can optionally include a needle 130 having a first end 131, a second end 133, and defining a lumen 135. The first end 131 can have any suitable shape configured to pierce and create access to the inflatable member 114. For example, the first end 131 can have a sharpened tip that can be tapered. The lumen 135 can be sized such that the coupling member 124 of the guidewire assembly 120 can be translated through the second end 123, through the lumen 135, and through the first end 121 of the needle 130. In some embodiments, the needle 130 can be inserted through a jejunum wall of the patient and through a sidewall of the inflatable member 114. The coupling member 124 and a portion of the guidewire 122 can then be translated through the lumen 135 of the needle 130 such that at least one of the coupling member 124 and a portion of the guidewire 122 is at least partially disposed within inflatable member 114. The needle 130 can then be removed from the inflatable member 114 via translating the needle 130 along the guidewire 122.

In some embodiments, the inflatable member 114 can be filled and/or inflated with a fluid (e.g., a liquid or a gaseous fluid) after being disposed in the jejunum of the patient. For example, the inflatable member 114 can be filled and/or inflated with a fluid and/or contrast medium such that the inflatable member 114 defines an echogenic space detectable using ultrasound imaging. In some embodiments, the inflatable member 114 can be filled and/or inflated with a contrast medium such that the location of the inflatable member 114 can be visualized using fluoroscopy. Inflating the inflatable member 114 can also increase the surface tension of the sidewall of the inflatable member such that the needle 130 and/or the guidewire 122 can more easily pierce the sidewall. Further, inflation of the inflatable member 114 can create a larger interior space within which the coupling member 124 can expand and/or be disposed. Inflation of the inflatable member 114 can also increase the target size of the inflatable member for visualization and targeting of the inflatable member 114 with the needle 130 and/or coupling member 124. In some embodiments, any portion of the sidewall of the inflatable member 114 can be pierced for engagement with a guidewire assembly 120 such that the engagement does not depend on a particular rotational orientation of the inflatable member 114.

In use, the inflation assembly 110 can be inserted through an orifice of a patient (e.g., a nose or mouth of a patient), through an esophagus of the patient, through a stomach of the patient, and into a jejunum of the patient such that the inflation member 114 is disposed with the jejunum of the patient and the elongated tube 112 extends from the inflation member 114 in the jejunum, through the stomach, through the esophagus, and out of the orifice of the patient. For example, the inflation assembly 110 can be threaded over a guidewire 118. Fluid can then be delivered to the inflatable member 114 via the inflation lumen 116. As described above, the fluid can include a fluid and/or contrast medium such that the inflatable member 114 is detectable via imaging (e.g., ultrasound or fluoroscopy). The inflatable member 114 can then be visualized such that the location of the inflatable member 114 can be identified.

While visualizing the location of the inflatable member 114, the guidewire assembly 120 can be inserted through a jejunum wall of the jejunum and coupled to the inflatable member 114. For example, the needle 130 can be inserted through the abdominal wall and jejunum wall of the patient and through a sidewall of the inflatable member 114 such that the first end 131 of the needle 130 (e.g., the tip) is disposed within the inflatable member 114. The coupling member 124 and a portion of the guidewire 122 can be inserted through the lumen 135 of the needle 130 and translated (e.g., pushed) through the lumen 135. The coupling member 124 can then be translated out from the first end 131 of the needle 130 such that the coupling member 124 is disposed within the inflatable member 114. The needle 130 can then be withdrawn from the patient via translation of the needle 130 relative to the coupling member 124 and the guidewire 122, leaving the coupling member 124 within the inflatable member 114 and the guidewire 122 extending through a wall of the inflatable member 114. Additionally, the inflatable member 114 can be deflated in preparation for being withdrawn in tandem with the coupling member 124 through the stomach and the esophagus.

In some embodiments, the elongated tube 112 can then be translated (e.g., pulled) through the orifice of the patient such that the inflatable member 114 translates the coupling member 124 and the guidewire 122. For example, the elongated tube 112 can be translated relative to the orifice until the inflatable member 114, the coupling member 124, and the first end 121 of the guidewire 122 have been translated through the stomach, through the esophagus, and out of the orifice and the guidewire extends from the first end 121, through the orifice, through the esophagus, through the stomach, into the jejunum and through the jejunum wall to the second end 123 disposed outside of the patient. Thus, the first end 121 of the guidewire 122 can extend from the nose or mouth of the patient and be accessible near the patient's head, and the second end 123 of the guidewire 122 can extend from the jejunum wall of the patient and be accessible near the patient's abdomen. In some embodiments, a feeding tube (not shown) can then be pushed over the first end 121 of the guidewire 122 and translated along the guidewire 122 through the esophagus, through the stomach, into the jejunum, and through the jejunum wall until a portion of the feeding tube is disposed outside of the patient near or on the skin of the patient and a portion of the feeding tube is disposed within the patient's jejunum. Then, the guidewire 122 can be removed from the patient via applying a retraction (e.g. pulling) force to the first end 121 of the guidewire 122 such that the second end 123 of the guidewire 122 is pulled through the jejunum wall of the patient, through the jejunum, the stomach, through the esophagus, and out of the patient's oral or nasal orifice. Alternatively, the guidewire 122 can be removed from the patient via applying a retraction force to the second end 123 of the guidewire 122 such that the first end 121 of the guidewire 122 is pulled through the patient's oral or nasal orifice, through the esophagus, through the stomach, through the jejunum, out of the jejunum wall, and out of the skin of the patient. Thus, the feeding tube can provide direct access to the jejunum of the patient such that food or liquid can be disposed in the jejunum via the feeding tube without traversing the esophagus. In some embodiments, rather than moving the coupling member 124 outside of the patient's body via the esophagus, the inflatable member 114 can be used to move the coupling member 124 to another region of the body (e.g., another region of the jejunum or outside of the jejunum).

In some embodiments, rather than threading a feeding tube along the guidewire 122 from a patient's nasal or oral orifice, through the esophagus, through the stomach, and into the jejunum, an end of a feeding tube can be inserted through the tract created through the abdomen wall and through the jejunum wall (e.g., over the guidewire 122) such that the feeding tube extends from the jejunum through the abdomen wall. For example, one or more dilators can be translated over the guidewire 122 through the abdomen wall to expand the diameter of the tract to receive the feeding tube. In some embodiments, one or more T-fasteners can be inserted through the tract created through the abdomen and through the jejunum wall and used to affix a portion of the jejunum to the abdomen wall.

As described herein, in some embodiments, the creation of a jejunostomy tract can be assisted with the external magnetic assembly 140. As described above, the magnetic assembly 140 can be configured to magnetically interact with the magnetic member 115 (i.e., attract magnetic member 115), such that magnetic member 115 of the inflation assembly 110 is urged toward the external magnetic assembly 140 against patient tissue disposed between the external magnetic assembly 140 and the magnetic member 115. In some embodiments, as described herein, the magnetic member 115 can be configured to include multiple segments and to be flexible. In an some embodiments, the magnetic member 115 can be located within a lumen passing through the inflatable member 114 and can be configured not to move substantially relative to the inflatable member 114. Thus, a movement of magnetic member 115 towards the magnetic assembly 140 results in a movement of inflatable member 114 towards the magnetic assembly 140. In some embodiments, a clinician can place an external magnet (i.e., external magnetic assembly 140) atop a patient's abdomen, which may attract the magnetic member 115 inside a patient's body, drawing the inflation assembly 110 including the inflatable member 114 superficially to the anterior abdominal wall. The external magnetic assembly 140 and the inflatable member 114 including the magnetic member 115 can thus be coapted, or brought together, though still separated by the intervening abdominal wall, adipose tissue, and dermal tissue planes. By coapting the tissue, the free-floating jejunal loop within which the inflatable member 114 and magnetic member 115 are disposed can be affixed in place via a temporary magnetic jejunopexy. These magnetic forces not only aid in drawing deep jejunal loops superficially, clearing away any intervening bowel or viscera, but also ensure that a portion of the jejunum is now fixed relative to the abdominal wall and no longer a “moving target” for needle access.

In some embodiments, after placing the external magnetic assembly 140, the inflatable member 114 can be filled with saline from the end of the elongated tube 112 outside the patient's mouth. Using ultrasound, the practitioner can assess the tissue between the skin and jejunum to ensure suitability for making the jejunostomy tract. The ultrasound can identify any intervening bowel, viscera, and vasculature that should be avoided. With a safe jejunostomy tract identified, the access needle can be inserted, aiming at the inflatable member 114. The needle path inside the body can be visualized real-time under ultrasound through the entire stomal tract, skin to jejunum. In addition to visual confirmation, aspirating fluid from inflatable member 114 will indicate and confirm when inflatable member 114 has been successfully accessed. Seeing the fluid from inflatable member 114 in the syringe lets the practitioner know that inflatable member 114 is safely penetrated and that a needle (e.g., needle 130) is not in an organ or other unintended anatomy. Once successful inflatable member 114 access is achieved, the proceduralist can finish placing the J-tube by standard methods employed in DPEJ and PRJ by snaring a guidewire via needle access or placing temporary T-fasteners and dilating the tract, respectively.

In some embodiments, magnetic jejunopexy first ensures the targeted lumen of jejunum is superficially located and maintains suitable attractive forces at up to 4.5 cm-150% deeper than the 3 cm tract depths at which DPEJ commonly fails. Second, ultrasound visualization from skin to lumen means that needle placement into a region of patient's body is well controlled. Proceduralists are able to track the path of needle 130's entry through tissue and directly into inflatable member 114, ensuring a placement of needle 130 that avoids vasculature and/or other organs. Critically, the magnetic jejunopexy provides an opposing force during needle stick that prevents movement of the jejunum. This is the most challenging step in the current standard of care, as the floating jejunum moves in multiple directions, and can collapse or even roll away upon needle insertion, leading to missed or through-and-through sticks that have the potential for patient harm. Finally, fluid aspiration provides positive confirmation of safe needle entry into the targeted lumen.

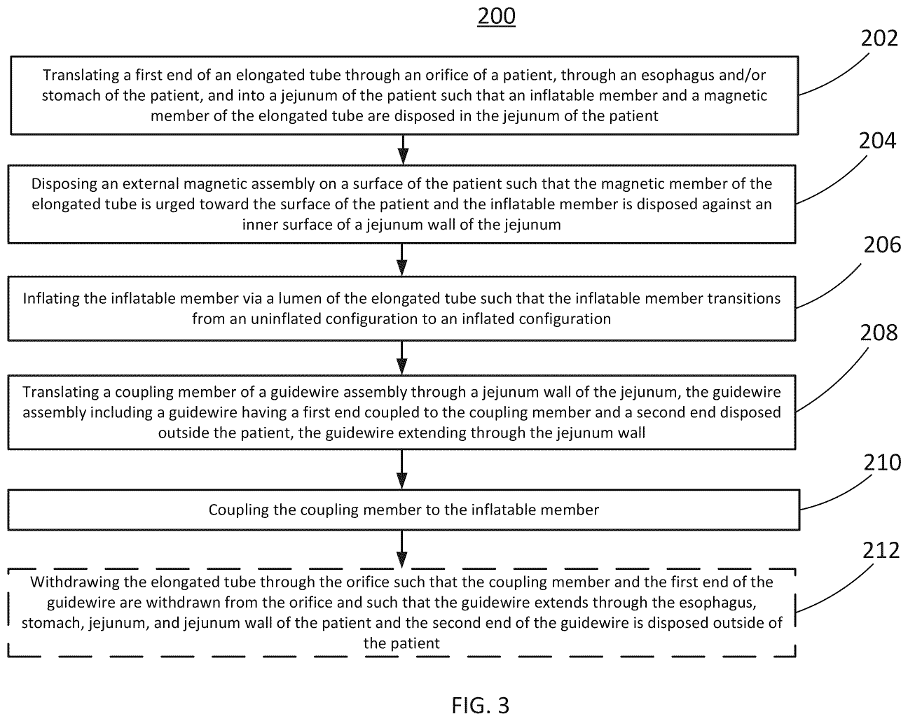

FIG. 3 is a flow chart of a method 200, according to an embodiment. The method 200 can be implemented using any of the systems or devices described herein, such as the system 100 described above. The method 200 includes, at 202, translating a first end of an elongated tube (e.g., the elongated tube 112) through an orifice of a patient, through an esophagus and/or stomach of the patient, and into the patient's jejunum such that an inflatable member (e.g., the inflatable member 114) and a magnetic member (e.g., the magnetic member 115) are disposed in jejunum.

At 204, an external magnetic assembly (e.g., the external magnetic assembly 140) is disposed on a surface of the patient (e.g., the patient's abdomen) such that the magnetic member of the elongated tube is urged toward the surface of the patient and the inflatable member is disposed against an inner surface of a jejunum wall of jejunum. In response to disposing the external magnetic assembly on the surface of the patient, the inflatable member and/or the magnetic member can urge an outer surface of the jejunum wall opposite the inner surface toward the surface of the patient such that a portion of the jejunum within which the inflatable member is disposed is fixed relative to the surface of the patient and urging the jejunum wall against tissue disposed between the portion of the jejunum and the surface of the patient (e.g., such that no air gaps are disposed between the inflatable member and the surface of the patient).

At 206, the inflatable member is inflated via a lumen of the elongated tube, such that the inflatable member transitions from an uninflated configuration to an inflated configuration. In some embodiments, a fluid (e.g., a liquid or a gaseous fluid) can be used to inflate the inflatable member. In some embodiments, the fluid can include a contrast medium and/or saline (e.g., a dyed saline, such as blue saline, may be used for a color confirmation as the blue colored liquid may not be confused with other bodily fluids). Optionally, the method can include visualizing the location of the inflatable member and/or a path from the surface of the patient to the inflatable member via ultrasound or fluoroscopy. Optionally, the method can include assessing, via ultrasound or fluoroscopy, any tissue or other intervening structures between the inflatable member and the surface (i.e., a potential path or tract to the jejunum and the inflatable member) and determining to extend a needle and/or guidewire through the surface of the patient, the tissue or other intervening structures, and the sidewall of the inflatable member if (e.g., only if) the visualized path to the inflatable member is suitable (e.g., the intervening tissue is coapted toward the surface, the path is free of undesirable intervening structures, and/or the intended portion of the jejunum is coapted). Optionally, if the path is determined to be unsuitable, the elongated tube and/or the external magnetic assembly can be adjusted (e.g., via advancing and/or retracting the elongated tube (e.g., using an endoscope as described herein) and/or moving the external magnetic assembly on the surface of the patient from a first location to a second location to move the inflatable member relative to the jejunum and urge a different portion of the jejunum toward the surface of the patient and/or urge the same portion of the jejunum toward the second location on the surface of the patient to avoid undesirable intervening structures. The new potential path between the new surface location and the inflatable member can then be assessed to determine if the potential path is suitable, similarly as described above.

At 208, the coupling member of a guidewire assembly is translated through a jejunum wall of a jejunum of the patient. The guidewire assembly can include a guidewire having first end coupled to coupling member and second end disposed outside of the patient. Thus, the coupling member of the guidewire assembly can be translated through the jejunum wall such that a portion of the guidewire extends through an opening of the jejunum wall, through any intervening tissue, and through the surface of the patient (e.g., along a path or tract as referenced above). The path or tract can be created by translating a needle along the path to the inflatable member (e.g., through a sidewall of the inflatable member). In some embodiments, the translation of the needle toward and into engagement with the inflatable member can be visualized in real-time to ensure that the needle tip reaches a target (e.g., within the inflatable member) and/or avoids undesirable intervening structures. Alternatively or additionally, in some embodiments, the needle can be advanced toward the inflatable member until a fluid flows (e.g., is aspirated) from the interior of the inflatable member, through a first end (e.g., the needle tip) of the needle, and out of the second end of the needle, confirming that the needle tip has reached the interior of the inflatable member. In some embodiments, fluid can be manually drawn from the needle during navigation of the needle through the patient and/or after the needle tip is believed to have reached the interior of the inflatable member to confirm that the needle tip is disposed within the inflatable member upon fluid flow through the needle of fluid known to be disposed within the inflatable member. As described herein, the confirmation fluid can be a non-naturally produced bodily fluid and/or have a color that is uncommon or atypical of fluid in a patient's body or in a healthy patient's body, such as blue). Upon disposing the needle tip within the inflatable member, the guidewire assembly can be advanced through the needle until the coupling member is disposed within the inflatable member or beyond the inflatable member (e.g., after being passed through a first sidewall of the inflatable member, through the interior of the inflatable member, and through a second sidewall of the inflatable member). In some embodiments, the needle may be advanced to an area near the inflatable member (e.g., through the jejunum portion but outside the inflatable member) and then the guidewire assembly may be advanced through and beyond the needle to engage the inflatable member.

At 210, the coupling member can be coupled with the inflatable member. For example, the coupling member can be engaged with one or more sidewalls of the inflatable member (e.g., from within the inflatable member and/or from a side of the inflatable member opposite the patient surface). For example, the coupling member can be transitioned from a first configuration to a second configuration in which the coupling member has a larger lateral extent or outermost diameter than the first configuration and the opening(s) in the inflatable member through which the coupling member has partially or fully translated through (e.g., by being translated from within a needle to beyond the needle as described above) such that the coupling member is retained by the inflatable member.

Optionally, at 212, the elongated tube can be withdrawn through the orifice in which the elongated tube was initially inserted, such that the coupling member and the first end of the guidewire are withdrawn from the orifice and such that the guidewire extends through the esophagus, stomach, jejunum, and jejunum wall of the patient and the second end of the guidewire is disposed outside of the patient.

In some embodiments, once the guidewire is placed within the patient, the proceduralist can finish placing the J-tube by standard methods employed in DPEJ and/or PRJ, respectively. It should be appreciated that steps 202-212 of the method 200 may vary. In some embodiments, the variation is due to various approaches used for delivering inflatable member 114 to the desired location (e.g., jejunum J). In some embodiments, at least some of the steps of the method 200 may be optional. For example, steps 208-212 of the method 200 may be optional.

In some embodiments, among multiple approaches for delivering the inflatable member to the desired location, a guidewire can be placed first. The guidewire can be guided from an orifice of the patient 100 (e.g., a mouth or nose) towards a desired destination (e.g., using an endoscope coupled with the guidewire). Subsequently, the inflation assembly including the elongated tube, the inflation member, and the magnetic member 115 can be guided via the guidewire to the desired destination (e.g., the jejunum).

In some embodiments, using another approach, an engagement portion of the elongated tube can be coupled directly to an endoscope. The endoscope can be configured to guide the elongated tube including the inflatable member to the desired destination. As described above, the engagement portion of the elongated tube and/or the endoscope can include structures to enable coupling of the endoscope to the elongated tube (e.g., via snaring or grasping of member).

In some embodiments, a guidewire may be placed first, and then an endoscope can tow the inflatable member of the elongated tube to the desired location, either over-the-guidewire or alongside the guidewire.

In some embodiments, an endoscope can define a lumen through which an inflation assembly including the elongated tube, inflatable member, and the magnetic member can be extended. The endoscope can deliver the inflatable member to a desired destination within the lumen. The endoscope can then be retracted relative to the inflatable member such that the inflatable member can be coupled with the coupling member of the guidewire and the endoscope can be withdrawn from the patient's body.

In some embodiments, the endoscope can be placed within the elongated tube such that elongated tube and inflatable member sheath the endoscope. Subsequently, the endoscope can guide the elongated tube and the inflatable member to the desired location, before being withdrawn relative to the elongated tube and removed from that location.

In some embodiments, each of the above approaches can be performed by inserting the endoscope through a mouth or nasal cavity of a patient. Alternatively, the endoscope may be guided through a gastrostomy providing access into a stomach of the patient, and the endoscope may be guided towards a location of the patient's body (e.g., towards jejunum J) from an orifice defined by the gastrostomy site. Alternatively, a guidewire may be guided through a gastrostomy providing access into a stomach of the patient, and the guidewire may be guided towards a location of the patient's body (e.g., towards jejunum J), potentially using imaging modalities such as fluoroscopy, from an orifice defined by the gastrostomy site. The elongated tube can then be placed over the guidewire placed distally into the small bowel (i.e. jejunum).

Other variations of the method 200 include variations for formation of a tract for placing a J-tube. In some embodiments, a needle can be guided to target the inflatable member. Once the inflatable member is pierced by the needle, a T-fastener or a similar device may be used to secure the jejunum to an abdominal wall of the patient.

In some embodiments, the needle can be used to pierce the inflatable member and to place a guidewire in engagement with the inflatable member (e.g., within the inflatable member), such that a coupling member coupled to the guidewire is “snared” by the inflatable member (e.g., by the inflatable member itself or by a built-in snare of the inflatable member). In some embodiments, when the endoscope is located in proximity of the inflatable member, a guidewire can be snared by the endoscopic snare. Further, as described above, the guidewire may be retracted through the access point (e.g., through a mouth of a patient) in tandem with the inflation assembly.

In some embodiments, a needle can be used to pierce the inflatable member or reach an area near the inflatable member and to place a guidewire in proximity to the inflatable member (e.g., within the inflatable member or near the inflatable member) such that the guidewire can be used to dilate a tract or passage to the jejunum from outside of abdomen of a patient (e.g., using serial dilation) for retrograde feeding tube placement.

In some embodiments, one or more T-fasteners (or similar retention devices) can be placed proximal or distal to the inflatable member using the inflatable member as a reference frame and/or endoscopic and/or fluoroscopic modalities for further visualization. The guidewire can be retracted through the access point or used for retrograde feeding tube placement.

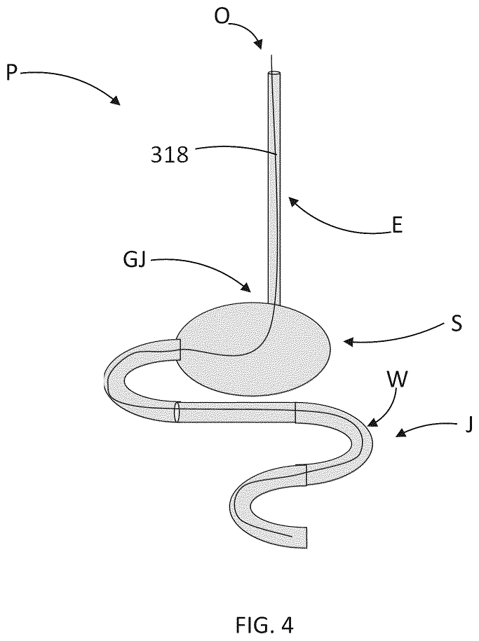

FIGS. 4-19 are schematic illustrations of a system 300 in various stages of operation. The system 300 can be the same or similar in structure and/or function to any of the systems or devices described herein, such as the system 100 described above. For example, the system 300 includes an inflation assembly 310 and a guidewire assembly 320. The system 300 also includes or can be associated with an external magnetic assembly 340 and an ultrasound probe 360. The inflation assembly 310 can include an elongated tube 312, an inflation member 314, and a magnetic member 315. The elongated tube 312 can have a first end 311 and a second end 313. In some embodiments, the elongated tube 312 can have a length sufficient to extend from at least an oral or nasal orifice of a patient to the jejunum of the patient. The inflatable member 314 and the magnetic member 315 can be coupled to the elongated tube 312 near the first end 311 of the elongated tube 312. The inflation assembly 310 can include an inflation lumen defined by the elongated tube 312 and in fluid communication with the inflatable member 314. The guidewire assembly 320 can include a guidewire 322 and a coupling member 324 disposed at an end of the guidewire 322.

As shown in FIG. 4, a guidewire 318 can be translated through an orifice O of a patient P (e.g., a nose or mouth of a patient), through an esophagus E of the patient, into a stomach S of the patient, and into a jejunum J of the patient such that a first end of the guidewire 318 is disposed within the jejunum J and a second end of the guidewire 318 is disposed exterior to the patient (e.g., near and/or extending from the orifice O).

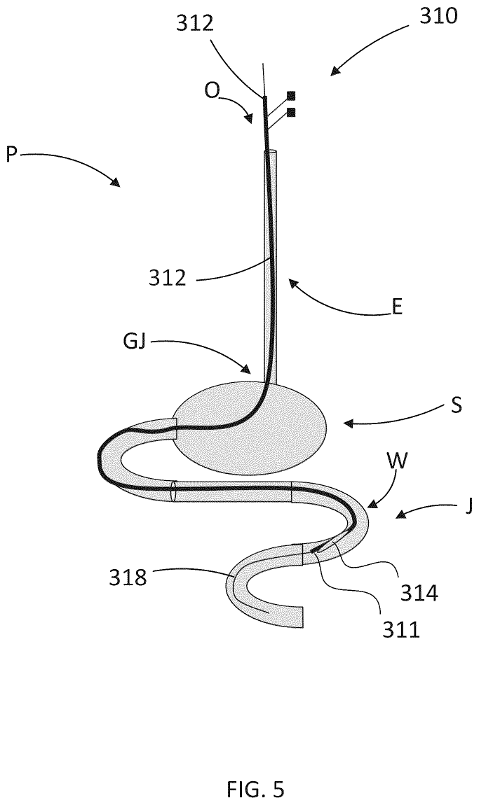

As shown in FIG. 5, an inflation assembly 310 can include an elongated tube 312 and an inflation member 314 disposed near a first end 311 of the elongated tube 312. A portion of the inflation assembly 310 can be inserted through an orifice O of a patient (e.g., a nose or mouth of a patient), through an esophagus E of the patient, into a stomach S, and into jejunum J of the patient such that the inflation member 314 is disposed within the jejunum J of the patient and the elongated tube 312 extends from the inflation member 314, through the esophagus E, and out of the orifice O of the patient. In some embodiments, as shown in FIG. 5, the elongated tube 312 may be guided along (e.g., threaded along) the guidewire 318. In some embodiments, a user can determine that the first end 311 of the elongated tube 312 is extended a particular distance into the jejunum J based on the known relative lengths of the tubular member 350) and the elongated tube 312 and/or markings on at least one of the tubular member 350) or the elongated tube 312. Although not shown in FIG. 5, in some embodiments, as described further herein, rather than being threaded along the guidewire 318, the elongated tube 312 may be translated (e.g., such that the inflation member 314 reaches the jejunum) by an endoscope coupled to an engagement portion of the inflation assembly 310 or threaded through a lumen of the elongated tube 312. In some embodiments, the guidewire 318 may not be used.

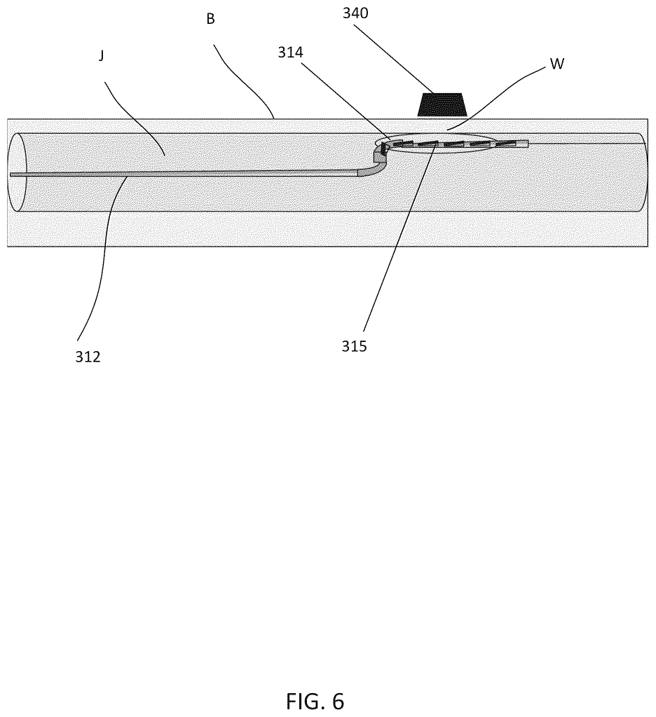

As shown in FIG. 6, the external magnetic assembly 340 can be placed on a surface of a patient (e.g., a surface of an abdomen B) such that the magnetic member 315 of the elongated tube 312 is urged toward the surface of the patient and the inflatable member 314 is disposed against an inner surface of a jejunum wall W of the jejunum J. In some embodiments, the magnetic member 315 can be urged toward the external magnetic assembly 340 such that the jejunum wall W is pulled toward and/or secured in place against abdomen tissues aligned with the external magnetic assembly 340 by the magnetic member 315. As shown in FIG. 6, in some embodiments, the magnetic member 315 can include a series of discrete magnetic segments (e.g., five magnetic segments). At least some of the magnetic segments can be disposed within or adjacent to the inflatable member 314 and/or the magnetic segments can be disposed symmetrically about a midpoint of the inflatable member 314 such that the inflatable member 314 contacts the inner surface of the jejunum wall W of the jejunum J in response to the external magnetic assembly 340 being placed on the surface of the abdomen B near or aligned with the location of the inflatable member 314. Alternatively, in some embodiments, a single magnetic segment can be used.

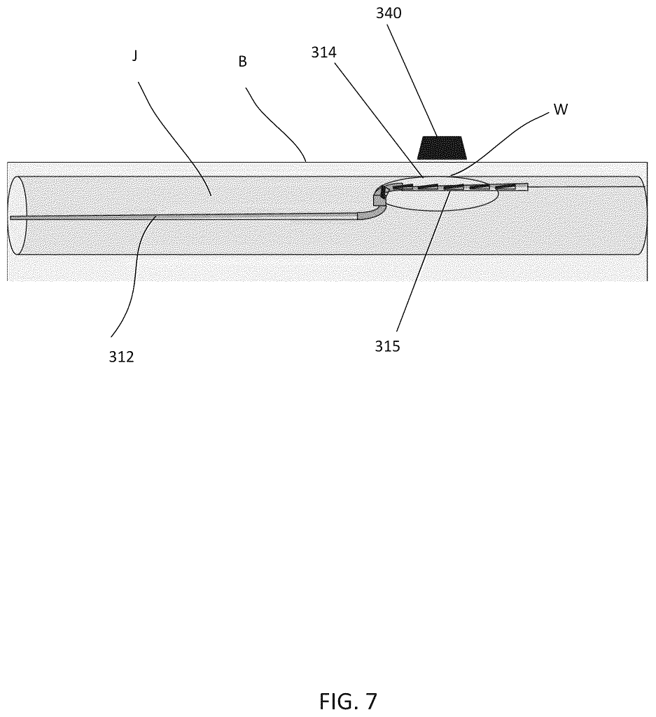

As shown in FIG. 7, fluid (e.g., a liquid or a gaseous fluid) can be delivered to the inflatable member 314 via an inflation lumen (not shown) of the elongated tube 312 such that the inflatable member 314 is filled and/or inflated. For example, the inflatable member 314 can be filled and/or inflated with a fluid and/or contrast medium such that the inflatable member 314 defines an echogenic space detectable using ultrasound imaging. In some embodiments, the inflatable member 314 can be filled and/or inflated with a contrast medium such that the location of the inflatable member 314 can be visualized using fluoroscopy. The inflatable member 314 can then be visualized using imaging (e.g., ultrasound or fluoroscopy) such that the location of the inflatable member 314 can be identified.

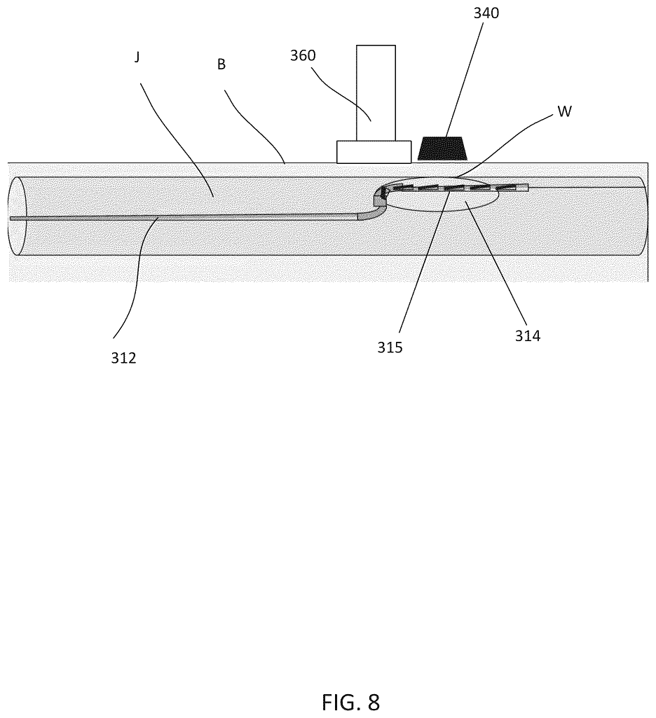

As shown in FIG. 8, the ultrasound probe 360 can be applied to the abdomen surface B of the patient P such that the inflatable member 314 and any intervening tissue or other structure between the inflatable member 314 and the surface of the abdomen B can be visualized and the location of the inflatable member 314 can be identified.

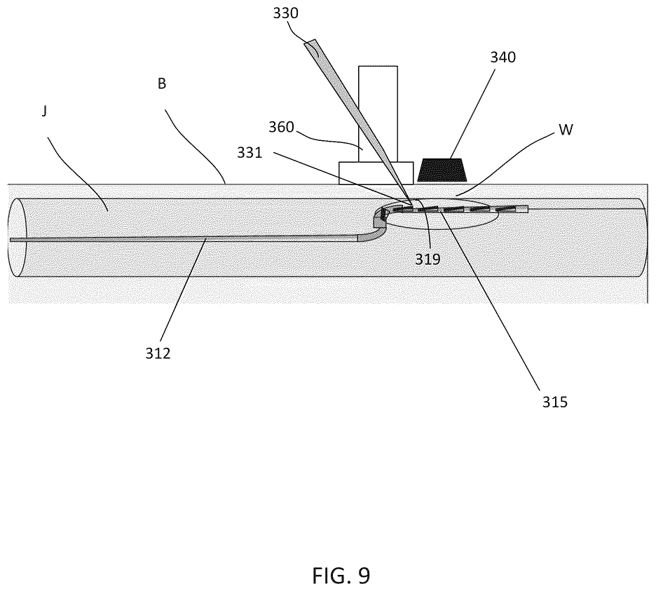

As shown in FIG. 9, while visualizing the location of the inflatable member 314 using the ultrasound probe 360, the needle 330 can be inserted through the abdomen B and the jejunum wall W of the patient and through a sidewall portion 319 of the inflatable member 314 such that a first end 331 of the needle 330 (e.g., the tip) is disposed within the inflatable member 314. During insertion of the needle 330, the ultrasound probe 360 can be used to visualize the needle 330 and any intervening patient structure between the skin of the patient and the inflatable member 314. Furthermore, the ultrasound probe 360) can be used to confirm that the first end 331 of the needle 330 is disposed within the inflatable member 314. Additionally, or alternatively, echogenic fluid (e.g., a dyed fluid, where a dye color may be selected to be different from colors of typical bodily fluids) can be aspirated from the inflatable member 314 via the needle 330 (e.g., into a syringe barrel) to verify that the first end 331 of the needle is disposed within the inflatable member 314.

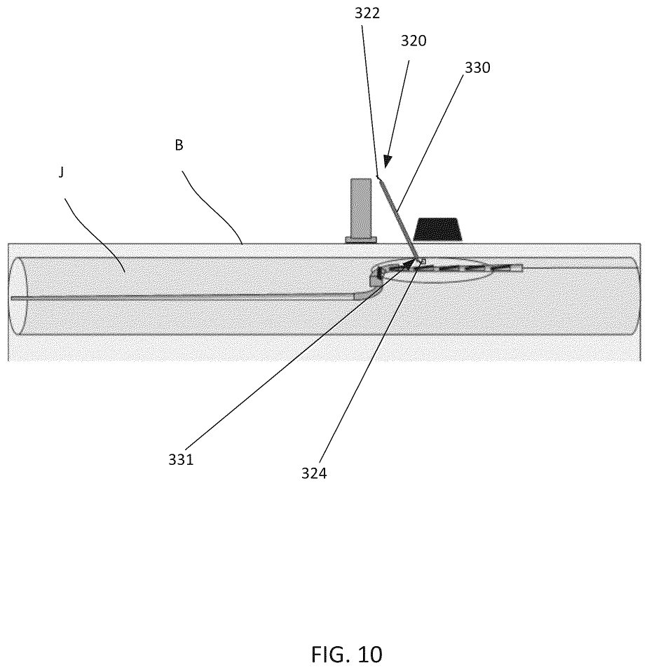

As shown in FIG. 10, with the first end 331 of the needle disposed within the inflatable member 314, the coupling member 324 and a portion of the guidewire 322 can be inserted through a lumen of the needle 330 and translated (e.g., pushed) through the lumen of the needle 330 until the coupling member 324 is disposed within the inflatable member 314. As shown in FIG. 10, the coupling member 324 can be configured to transition to a pigtail shape when translated out of the first end 331 of the needle 330. Although shown as transitioning to a pigtail shape, the coupling member 324 can be configured to transition to any suitable shape, such as, for example, a hook, a coil, or a corkscrew shape.

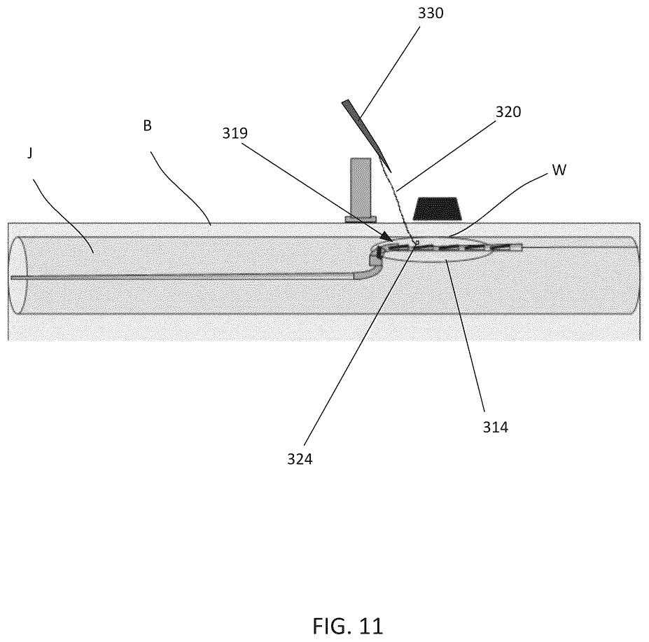

As shown in FIG. 11, the needle 330 can then be withdrawn from the patient via translation of the needle 330 relative to the coupling member 324 and the guidewire 322, leaving the coupling member 324 within the inflatable member 314 and the guidewire 322 extending through the wall portion 319 of the inflatable member 314, the jejunum wall W, and the abdomen B of the patient P. Additionally, the external magnetic assembly 340 can be removed from the patient such that the magnetic member 315 is no longer urged (e.g., via magnetic attraction) toward the anterior tracheal wall. The inflatable member 314 can be deflated in preparation for being withdrawn through the esophagus.

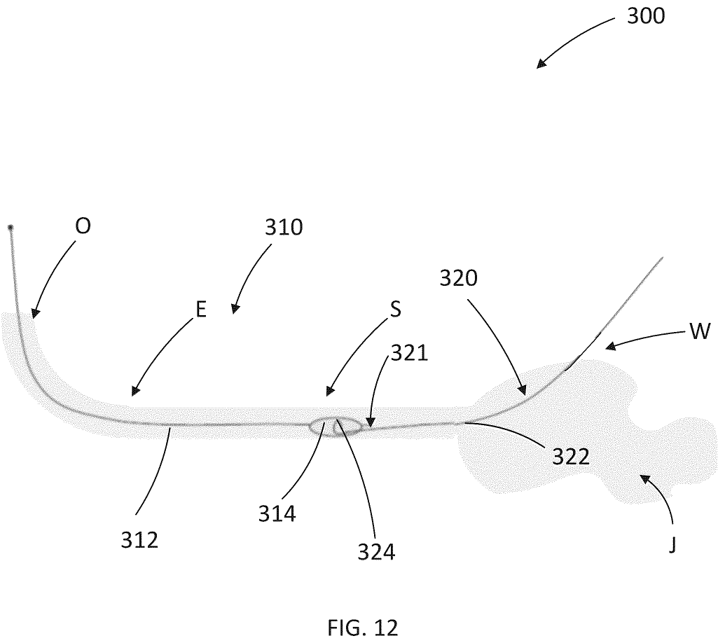

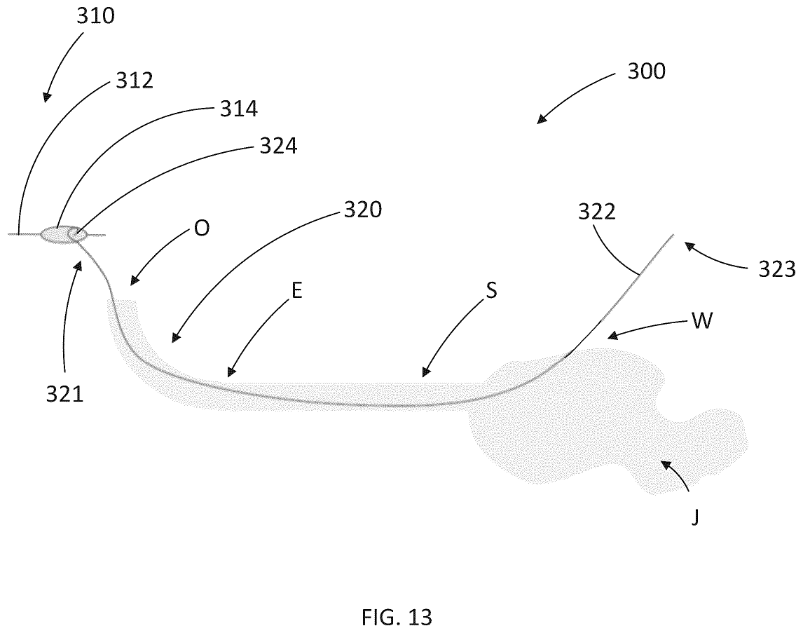

With the guidewire assembly 320 extending from the interior of the jejunum J through the abdomen B of the patient P and coupled to the inflatable member 314, any suitable percutaneous jejunostomy procedure can be performed using the guidewire assembly 320 and the tract through the patient's abdomen B to the patients jejunum J through which the guidewire assembly 320 is disposed. For example, it may be desired to translate the coupling member 324 of the guidewire assembly 310 (e.g., for use of the guidewire 322 for introduction of a jejunostomy tube along the guidewire 312, into the jejunostomy J, and out of the abdomen B through the tract within which the guidewire 322 is disposed). For example, as shown in FIG. 12, the elongated tube 312 can then be translated (e.g., pulled) through the orifice O of the patient such that the inflatable member 314 translates the coupling member 324 and the guidewire 322 in tandem. For example, the elongated tube 312 can be translated relative to the orifice O until the inflatable member 314, the coupling member 324, and a first end 321 of the guidewire 322 have been translated through the stomach S and the esophagus E (as shown in FIG. 12) and out of the orifice O (as shown in FIG. 13), such that the guidewire 322 extends from the location of the coupling member 324 outside of the patient, through the orifice O, through the esophagus E, into the stomach S, into the jejunum J, and through the jejunum wall W to a second end 323 of the guidewire 322 disposed outside of the patient. Thus, the first end 321 of the guidewire 322 can extend from the nose or mouth of the patient and be accessible near the patient's head, and the second end 323 of the guidewire 322 can extend through the jejunum wall W of the patient and be accessible near the patient's abdomen.

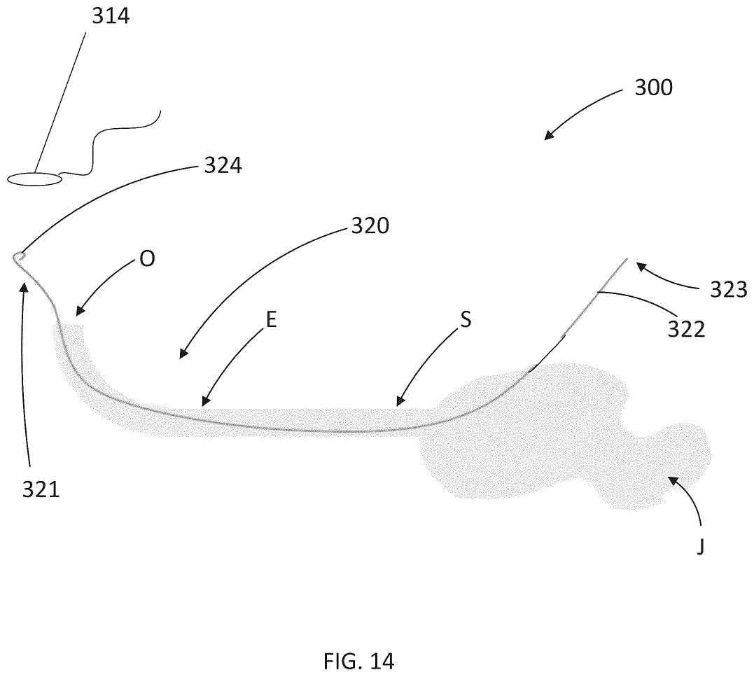

As shown in FIG. 14, the inflatable member 314 can then, optionally, be decoupled from the coupling member 324 of the guidewire assembly 320 at end 321. For example, a user can apply a first force on the inflatable member 314 and a second force on the coupling member 324 in an opposite direction from the first force. The opposite forces can cause the pigtail-shaped coupling member 324 to straighten and decouple from the inflatable member 314 (e.g., slide out of the orifice in the inflatable member 314 created by the needle 330 within which the guidewire 322 is disposed).

As shown in FIG. 15, the coupling member 324 can be separated from the rest of the guidewire assembly 320 so that the coupling member 324 does not impede a feeding tube (e.g., a jejunostomy tube) from being threaded along the guidewire 322. For example, the guidewire assembly 320 can be cut adjacent the coupling member 324 near the first end 321 of the guidewire 322 so that the coupling member 324 can be removed from the guidewire 322. The user can then dispose of the coupling member 324. In some embodiments, rather than decoupling the coupling member 324 from the guidewire assembly 320 prior to separating the coupling member 324 from the remainder of the guidewire assembly 320, the inflatable member 314 and the coupling member 324 can remain engaged during the separation of the coupling member 324 from the remainder of the guidewire assembly 320. The user can then dispose of the coupling member 324 and the inflatable member 314 simultaneously,

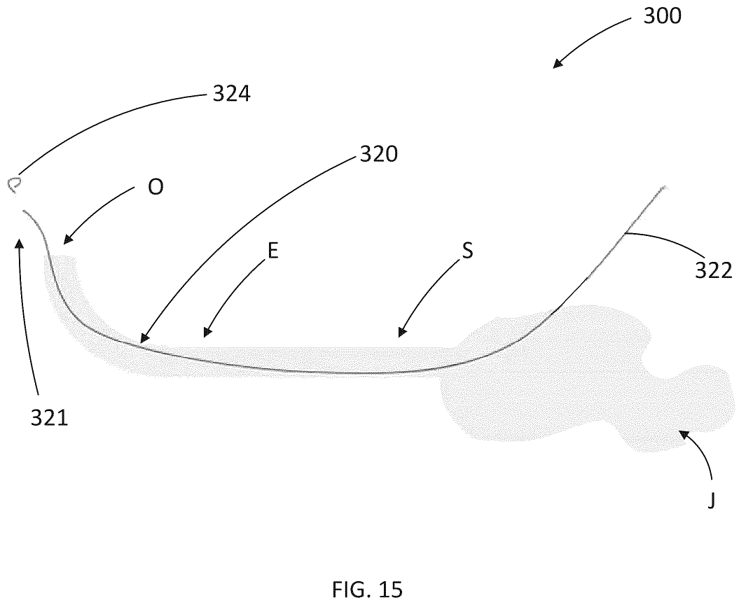

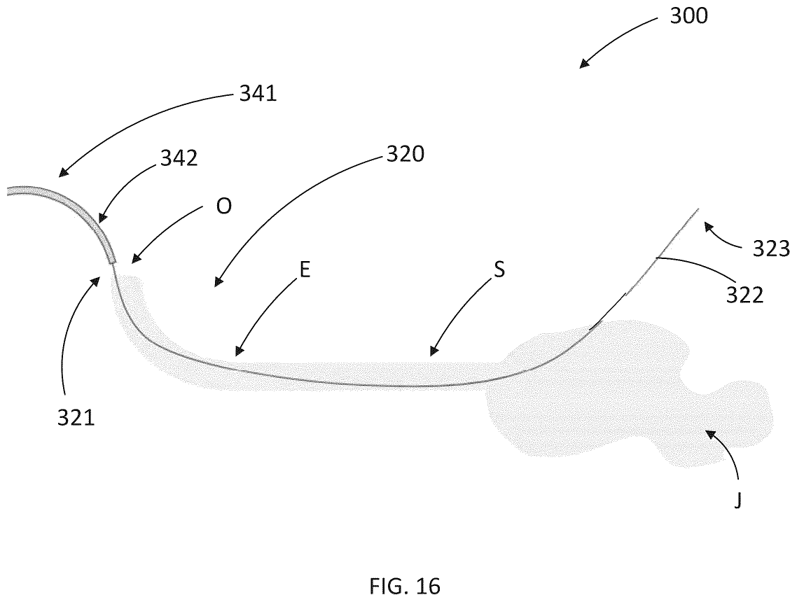

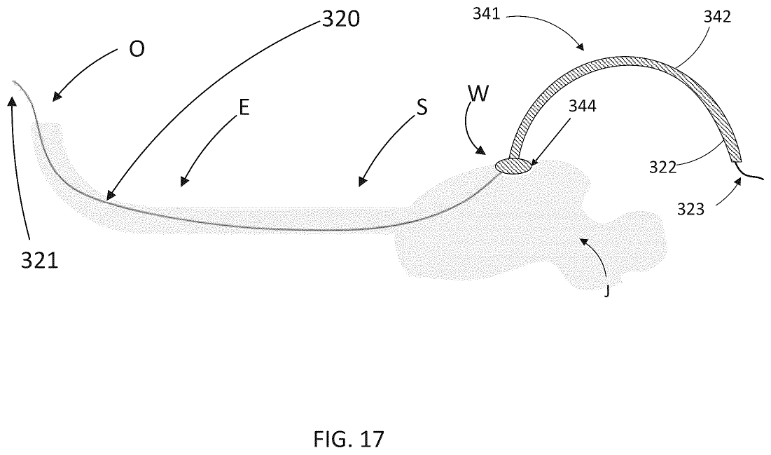

As shown in FIG. 16, a feeding tube 341 can then be pushed over the first end 321 of the guidewire 322 and translated along the guidewire 322 through the esophagus, into the stomach, into the jejunum, and through the jejunum wall until a portion of the feeding tube 341 is disposed outside of the patient near or on the skin of the patient and a portion of the feeding tube 341 is disposed within the patient's jejunum. For example, as shown in FIG. 17, the feeding tube 341 can include a tube portion 342 and a retention portion 344. The retention portion 344 can be engaged with the inner surface of the wall W of the patient's jejunum J and the tube portion 342 can extend through the patient's jejunum wall W and outside of the patient. The guidewire 322 can then be removed from the patient via applying a retraction (e.g. pulling) force to the first end 321 of the guidewire 322 such that the second end 323 of the guidewire 322 is pulled through the jejunum wall W of the patient, through the jejunum J, the stomach S, through the esophagus E, and out of the patient's oral or nasal orifice O, leaving the feeding tube 341 in place extending through the jejunum wall W. Alternatively, the guidewire 322 can be removed from the patient via applying a retraction force to the second end 323 of the guidewire 322 such that the first end 321 of the guidewire 322 is pulled through the patient's oral or nasal orifice O, through the esophagus E, through the stomach S, the jejunum J, out of the jejunum wall W, and out of the skin of the patient (e.g., through the abdomen). Thus, the feeding tube 341 can provide direct access to the jejunum of the patient such that food or liquid can be disposed in the jejunum via the feeding tube 340 without traversing the esophagus.

Although the system 300 shows the coupling member 324 disposed within the interior of the inflatable member 314, in some embodiments the coupling member 324 can be disposed outside of the inflatable member 314 and coupled to an outer surface of the inflatable member 314 when the coupling member 324 and the inflatable member 314 are coupled to each other. For example, the coupling member 324 can have any suitable shape and can be coupled to the inflatable member 314 via any of the arrangements shown and/or described in the 441 Application.

In some embodiments, rather than threading a feeding tube along a guidewire 312 from the oral or nasal orifice O to the jejunum J, a T-fastener can be disposed within the tract through the abdomen B and the jejunum wall W through which the guidewire 312 is disposed. For example, as shown in FIG. 18, a T-fastener 391 may be inserted into the jejunum J over the guidewire 312, when guidewire 312 exits the jejunum J outside the abdomen B of the patient. In some embodiments, a position of a first portion 392 of the T-fastener 391 relative to a remaining portion of the T-fastener 391 can be adjusted (e.g., between a first position aligned with the guidewire 312 for insertion through the tract of the guidewire 312 to a second position perpendicular to the guidewire 312 and parallel to the surface of the abdomen B), thus allowing for the first portion 392 of the T-fastener 391 to enter the jejunum J in the first position and be adjusted to the second position such that the first portion 392 is anchored within the jejunum J. In some embodiments, the T-fastener 391 can fasten a portion of the wall W of jejunum J to the abdominal wall B. As previously described, the ultrasound assembly 360 may be configured to assist in placing one or more T-fasteners 391. In some embodiments, the inflation assembly 310 including the elongated tube 312, the inflatable member 314, and the magnetic member 315 may be present during the installation of the T-fastener 391, and in other embodiments, the inflation assembly 310 may already be removed, and only the guidewire 312 may be used for guiding the T-fastener 391 relative to the tract within which the guidewire 312 is disposed.

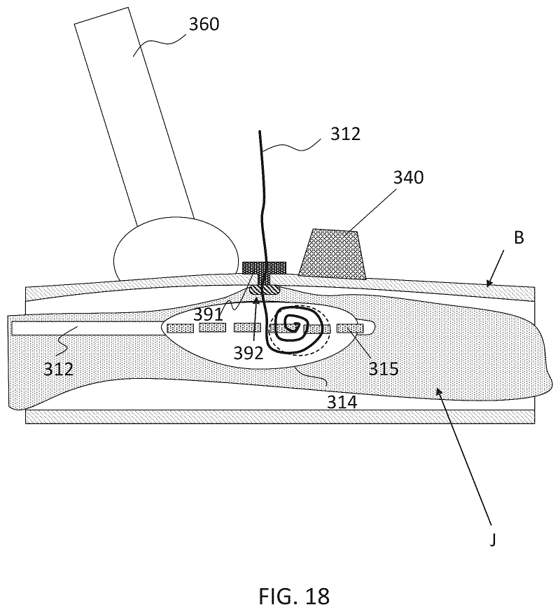

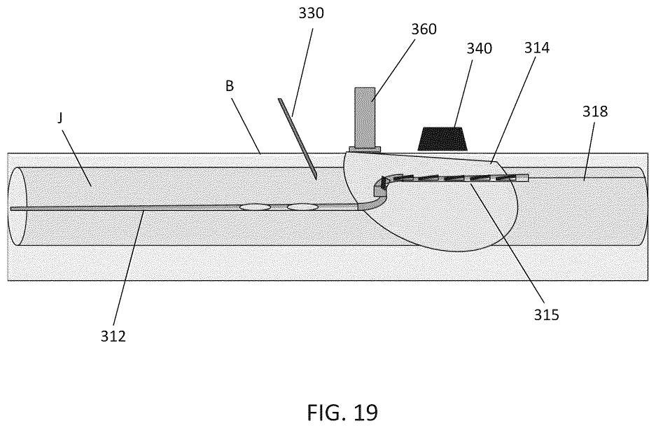

In some embodiments, rather than piercing the inflatable member 314 with the needle 330, the inflatable member 314 can be used to stabilize and/or distend a portion of the jejunum J and the end (e.g., tip) of the needle 330 can be placed within the jejunum using fluoroscopy or endoscopic visualization. For example, as shown in FIG. 19, the external magnetic assembly 340) and the magnetic member 315 can be used to create a temporary magnetic jejunopexy to stabilize the jejunum J relative to the abdomen wall B. The needle 330) can be inserted adjacent to (e.g., proximal to) the inflatable member 314. In some embodiments, as shown in FIG. 19, the inflatable member 314 may be sufficiently large (e.g., have a sufficiently large diameter and/or perimeter) to exert pressure on the surrounding walls of jejunum J in the inflated configuration (e.g., even in the absence of magnetic engagement between the external magnetic assembly 340 and the magnetic member 315). In some embodiments, a size and/or dimension of the inflatable member 314 can be calibrated or selected for a particular patient (e.g., based on a patient's age, patient's gender, or patient's size). A guidewire (not shown) can then be threaded through the needle 330 and used for placement of a feeding tube and/or a T-fastener 391 through the tract of the guidewire (e.g., after dilation of the tract).

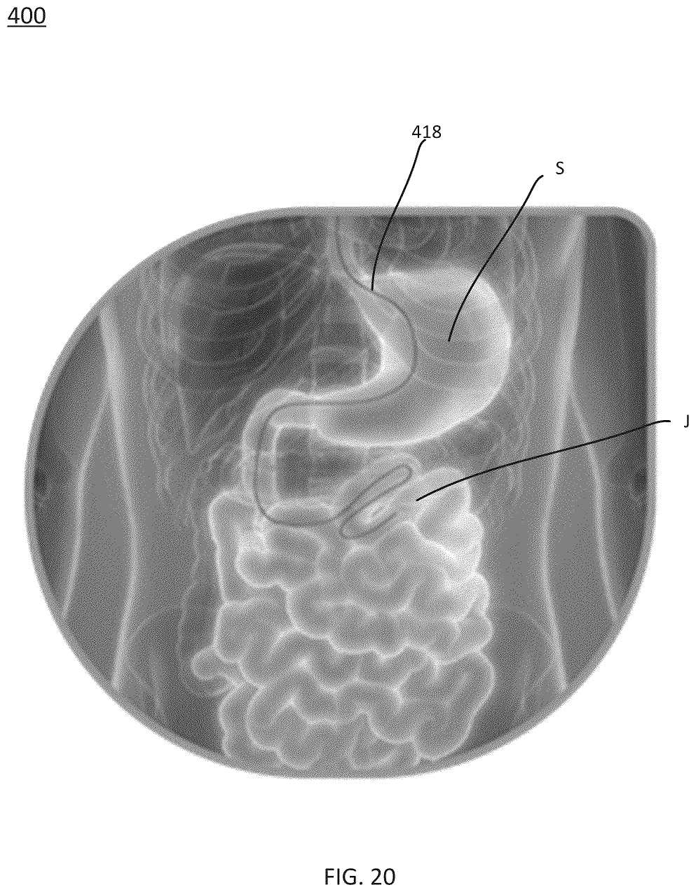

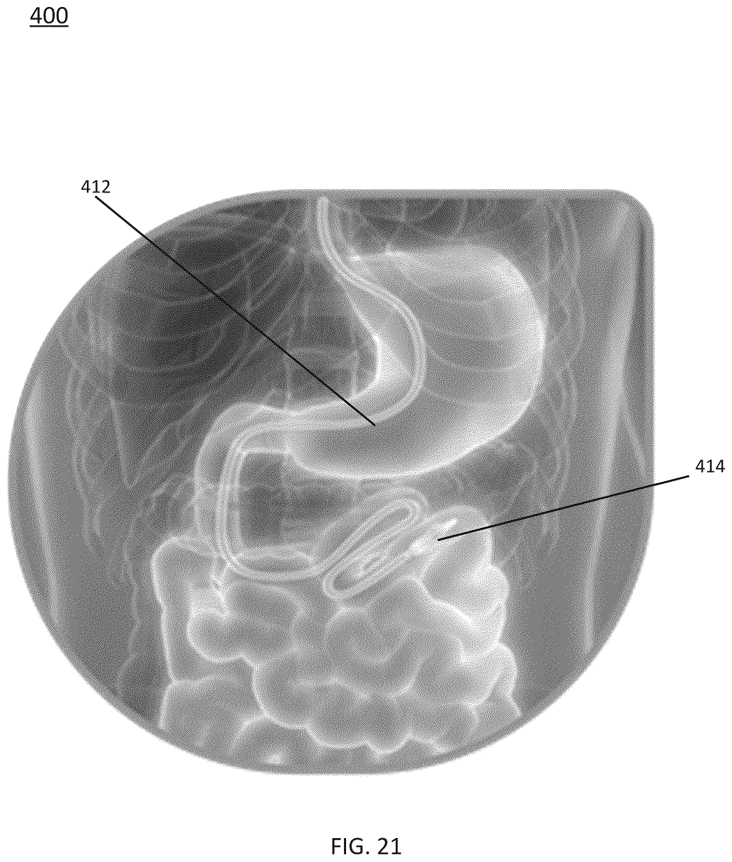

FIGS. 20-23 are schematic illustrations of a system 400 in various stages of operation. The system 400 can be the same or similar in structure and/or function to any of the systems described herein, such as the system 100 and/or the system 300. In some embodiments, under fluoroscopic guidance, a guidewire 418 (which may be the same or similar in structure and/or function to any of the guidewires described herein, such as the guidewire 118) is fed orogastrically through the upper GI tract, enabling the proceduralist to track its path, past the ligament of Treitz and into the proximal jejunum J, as shown in FIG. 20. An elongated tube 412 (e.g., a sterile catheter) (which may be the same or similar in structure and/or function to the any of the elongated tubes described herein, such as the elongated tube 112) coupled with an inflatable member 414 (which may be the same or similar in structure and/or function to any of the inflatable members described herein, such as the inflatable member 414) is introduced over the wire (e.g., using hand-driven force) and guided such that the inflatable member 414 reaches an appropriate location within the bowel, as shown in FIG. 21. In some embodiments, as described herein, a distal end of the elongated tube 412 may be carried into the jejunum J by aid of an endoscope. The endoscope can couple to the inflatable assembly 410 in a variety of ways, including mechanical grasping, magnetic attachment, and/or via an endoscope working channel or sheathing, as further described below.

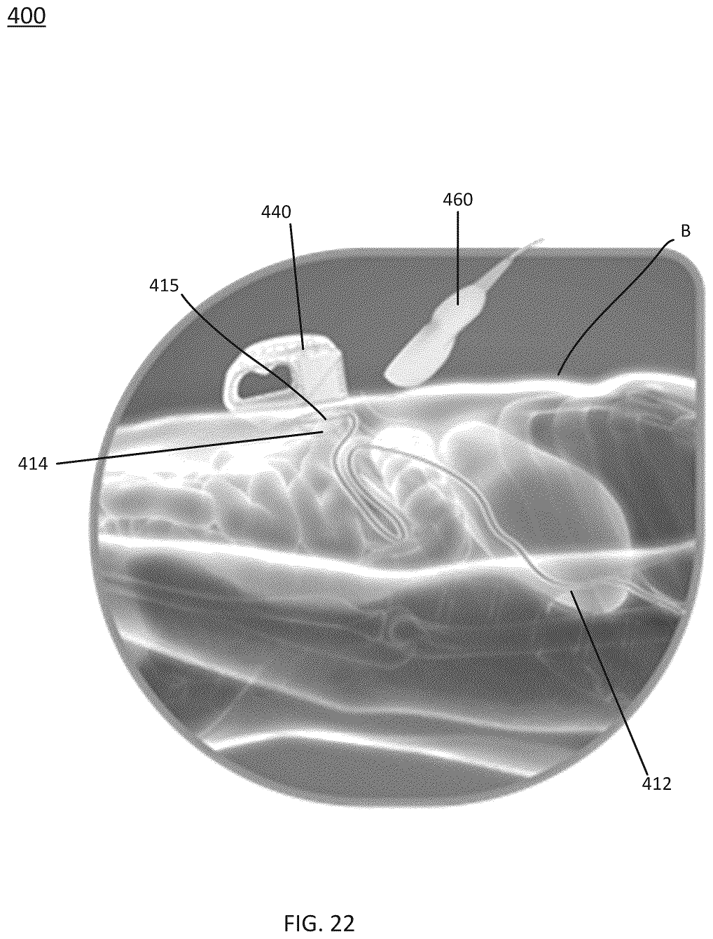

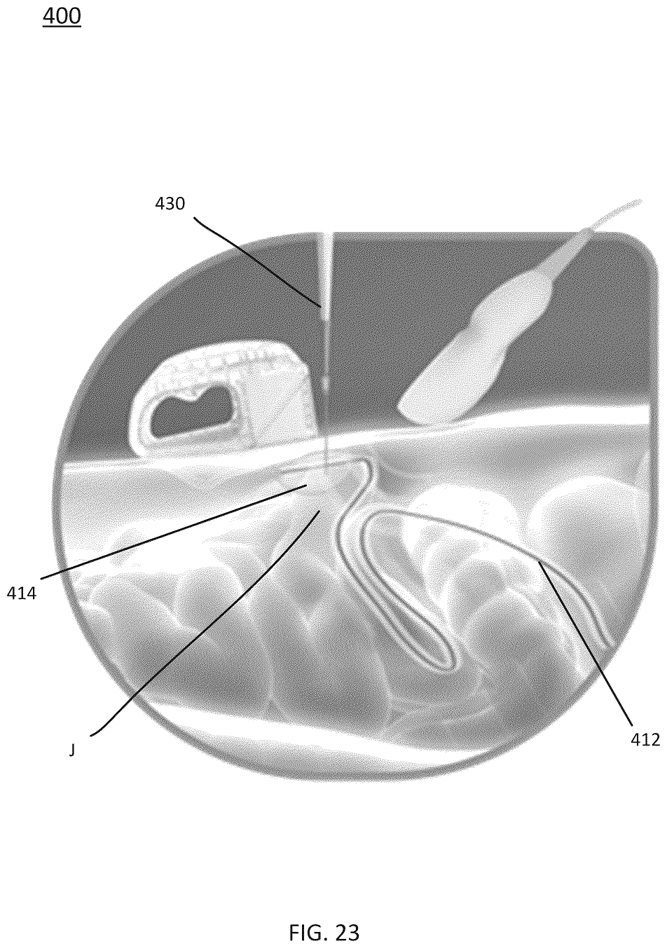

As shown in FIG. 22, when the inflatable member 414 is disposed at a desired location within the jejunum J, an external magnetic assembly 440 can be disposed on an abdominal surface B of the patient such that a magnetic member 415 coupled to the elongated tube 412 and/or the inflatable member 414 is urged toward the external magnetic assembly 440. The external magnetic assembly 440 and the magnetic member 415 can be the same or similar in structure and/or function to any of the external magnetic assemblies and magnetic members described herein, respectively. An ultrasound probe 460 (which may be the same or similar in structure and/or function to any of the ultrasound probes described herein) may be used to visualize placement of a needle 430 relative to the patient's anatomy as shown in FIG. 23. For example, using ultrasound visualization, a practitioner may assess tissue between the skin of the abdomen wall B and the jejunum J to ensure suitability for making the jejunostomy tract. Ultrasound visualization can be used to identify any intervening bowel, viscera, and/or vasculature that should be avoided during the translation of the needle 430. With a safe jejunostomy tract identified, the access needle (e.g., needle 430)) can be inserted, aiming at the inflatable member 414 as shown in FIG. 23.

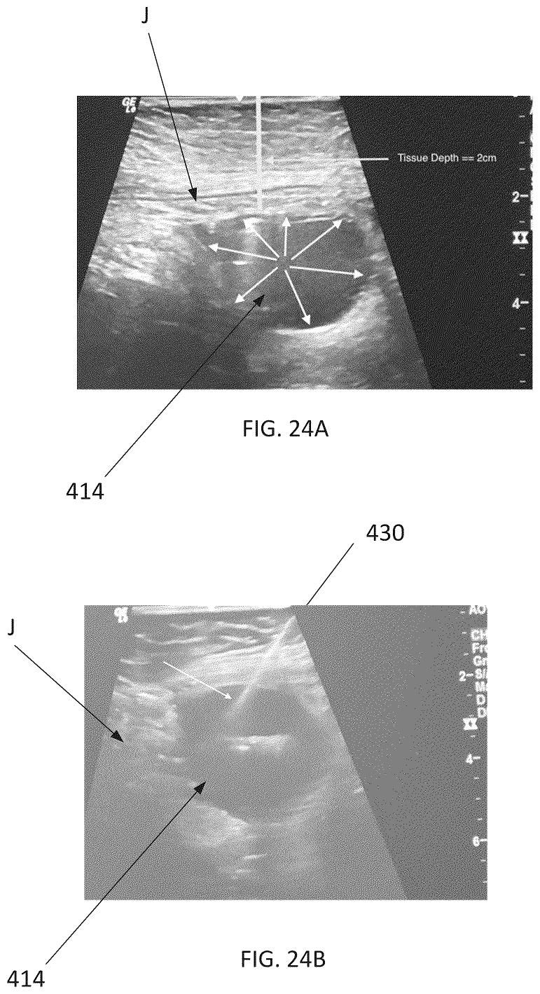

FIG. 24A is an ultrasound image of the inflatable member 414 within the patient's jejunum J. FIG. 24B is an ultrasound image of the needle 430 contacting the inflatable member 414 within the patient's jejunum J. As shown, the path of the needle 430 inside the body can be visualized real-time under ultrasound through the entire stomal tract, skin to jejunum as shown in FIG. 24. In addition to visual confirmation using ultrasound, fluid can be aspirated from the inflatable member 414 through the needle 430 to indicate and confirm that the needle 430) has successfully pierced the inflatable member 414. Seeing the fluid from the inflatable member 414 in a syringe coupled to the needle 430 allows the practitioner to know that the inflatable member 414 has been penetrated safely and that the needle 430 has not penetrated an organ or other unintended anatomy rather than the inflatable member 414.

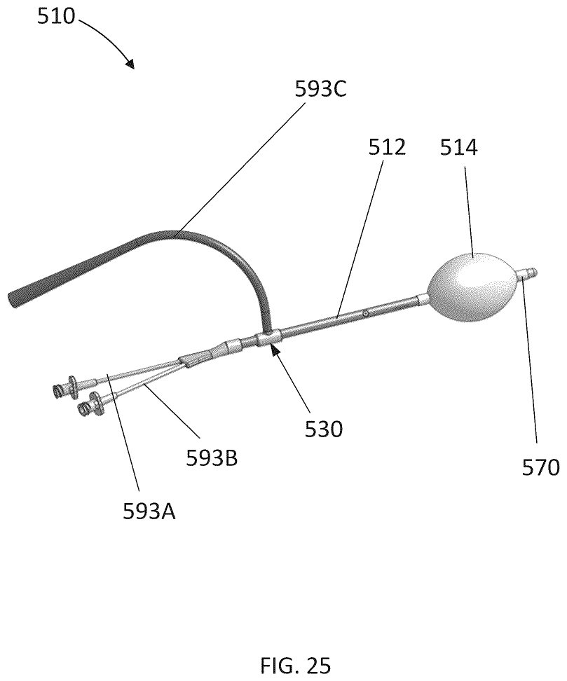

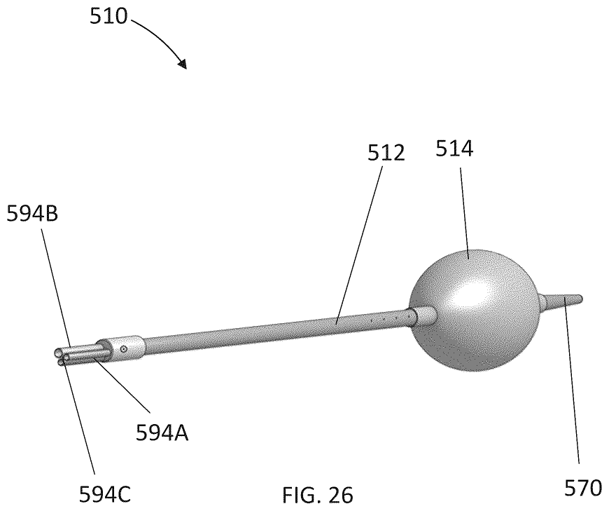

FIGS. 25-27 are various views of an inflation assembly 510. FIG. 25 is a side view of the inflation assembly 510, FIG. 26 is a side view of the inflation assembly 510 with a proximal portion removed to expose internal lumens of the inflation assembly 510, and FIG. 27 is a perspective view of the inflation assembly 510 with an elongated tube 512 and inflatable member 514 shown as being transparent such that the magnetic member 515 is visible. The inflation assembly 510 can be the same or similar in structure and/or function to any inflation assembly described herein, such as the inflation assembly 310. For example, the inflation assembly 510 includes an elongated tube 512, an inflatable member 514, and an engagement portion 570. Additionally, the inflation assembly 510 includes ports 593A-C in fluid communication with lumens 594A-C within the elongated tube 512, respectively. The first lumen 594A can be, for example, a guidewire lumen. The guidewire lumen can extend from a proximal end through a distal end of the inflation assembly 510. The second lumen 594B can be, for example, an insufflation lumen. In some embodiments, elongated tube 512 may include openings for allowing insufflation fluid to be delivered from elongated tube 512 to a body cavity in which elongated tube 512 is inserted. The third lumen 594C can be, for example, an inflation lumen for inflating the inflatable member 514. In some embodiments, the engagement portion 570) can be configured to engage with an endoscope 550, as further described below. In some embodiments, the engagement portion 570) can include and/or be coupled to various sensors such as a camera or any other suitable sensors. The engagement portion 570 can have any suitable shape, such as the rounded shape shown in FIG. 25 (e.g., configured to engage with or be coupled to an endoscope) or the tapered shape shown in FIG. 27 and defining a central guidewire lumen.

As shown in FIG. 27, the magnetic member 515 can include discrete magnetic segments. The magnetic member 515 can be positioned within (e.g., in a middle section of) the inflatable member 714 (e.g., within the elongated tube 512). Having multiple magnetic segments or elements allows for a flexible magnetic member 515, as described above with respect to the magnetic member 115. As shown, in some embodiments, each the discrete magnetic segments can contact an adjacent magnetic segment, and the magnetic segments can be serially arranged and freely movable relative to the adjacent magnetic segments. Although FIG. 27 shows four discrete magnetic segments, any suitable number of magnetic segments can be included. In some embodiments, the magnetic member 515 can have an overall length substantially equal to or less than the length of the inflatable member 514 (taken along a central axis of the elongated tube 512). For example, the overall length of the magnetic member 515 (e.g., a distance from a distalmost portion of a distalmost discrete magnetic segment and a proximalmost portion of a proximalmost magnetic segment) can be about 100%, about 95%, about 90%, about 80%, about 70%, about 60%, about 50%, about 40% between about 95% and about 100%, between about 90% and about 100%, between about 80% and about 100%, between about 70% and about 100%, between about 60% and about 100%, between about 50% and about 100%, and/or between about 85% and about 95% of the overall length of the inflatable member 514. In some embodiments, the overall length of the magnetic member 515 can be longer than the overall length of the inflatable member 515. In some embodiments, the magnetic member 515 can be centered relative to a first and second end of the inflatable member 515. In some embodiments, the magnetic member 515 can extend the same length distally of the inflatable member 515 as proximally of the inflatable member 515. In some embodiments, the discrete magnetic segment can be spaced apart from adjacent magnetic segments when the magnetic member 515 is in a straight orientation similarly as shown in FIG. 27 and/or when the magnetic member 515 is in a flexed orientation (e.g., including one or more curves). In some embodiments, the discrete magnetic segments can be equally spaced apart and/or symmetric about a center point of the inflatable member 514 and/or a center point of the portion of the elongated tube 512 disposed within or directly coupled to the inflatable member 514.

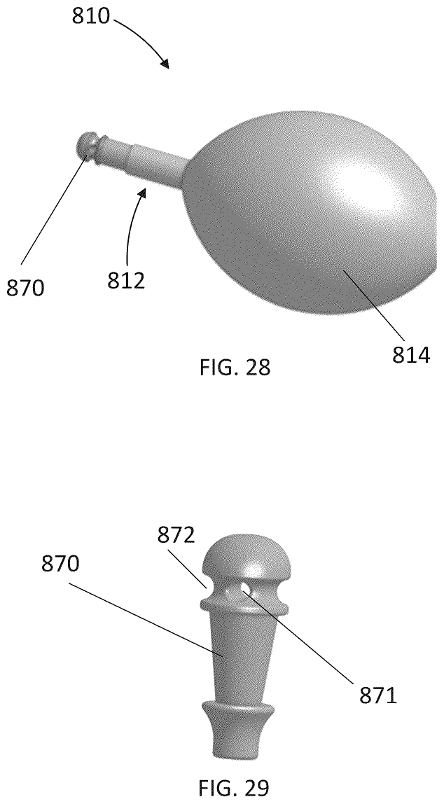

FIG. 28 is a perspective view of a distal end of an inflation assembly 810. The inflation assembly 810 can be the same or similar in structure and/or function to any of the inflation assemblies described herein. For example, the inflation assembly 810 can include an inflatable member 814 and an engagement portion 870 on a distal end of an elongated tube 812. FIG. 29 is a perspective view of the engagement portion 870. The engagement portion 870 can be the same or similar in structure and/or function to any of the engagement portions described herein. As shown in FIG. 29, the engagement portion 870 defines a circumferential groove 872 and through-hole 871. The circumferential groove 872 can be coaxial with a central axis of the engagement portion 870 and/or the distal or first end of an elongated tube 812. Although the engagement portion 870 defines both a circumferential groove 872 and a through-hole 871, in some embodiments, the engagement portion 870 can include only the circumferential groove 872 or the through-hole 871. As shown in FIG. 29, in some embodiments, the through-hole 871 can be defined between a first portion of the circumferential groove 872 and a second portion of the circumferential groove 872 (e.g., between opposite portions of the circumferential groove 872). In some embodiments, the through-hole 871 can be disposed at a non-zero angle (e.g., perpendicular or otherwise angled but not concentric) relative to a central axis of the engagement portion 870 and/or first or distal end of the elongated tube 812. Although the circumferential groove 872 is shown as being continuous, in some embodiments it is formed of discrete sections. Both the circumferential groove 872 and the through-hole 871 can be coupled to a flexible loop element (e.g., associated with or couplable to an endoscope), as further described below. The engagement portion 870 can include a rounded distal tip, which can be adjacent to the circumferential groove 872 as shown in FIG. 29. Additionally, the engagement portion can include a tapered portion that decreases in diameter along a central axis of the engagement portion (e.g., a proximal portion of the tapered portion has a smaller diameter than a distal portion of the tapered portion) and/or an end portion having a wider distal portion than proximal portion with a curved profile, as shown in FIG. 29. The proximalmost portion of the tapered portion can have a smaller diameter than the distal portion of the end portion. The distalmost portion of the tapered portion can have a smaller diameter than a portion of the engagement portion 870 forming a proximal portion of the circumferential groove 872. Thus, the tapered portion can be disposed between proximal and distal flanges. In some embodiments, the tapered portion is not tapered and/or not partially conical, and can have any suitable shape. In some embodiments, the distal flange formed at the intersection of the portion of the engagement portion 870 defining the circumferential groove 872 and the tapered portion can be configured to mate with the distal end surface of the elongated tube 812 and the tapered portion and end portion are configured to be disposed within an interior (e.g., within a central lumen) of the elongated tube 812.

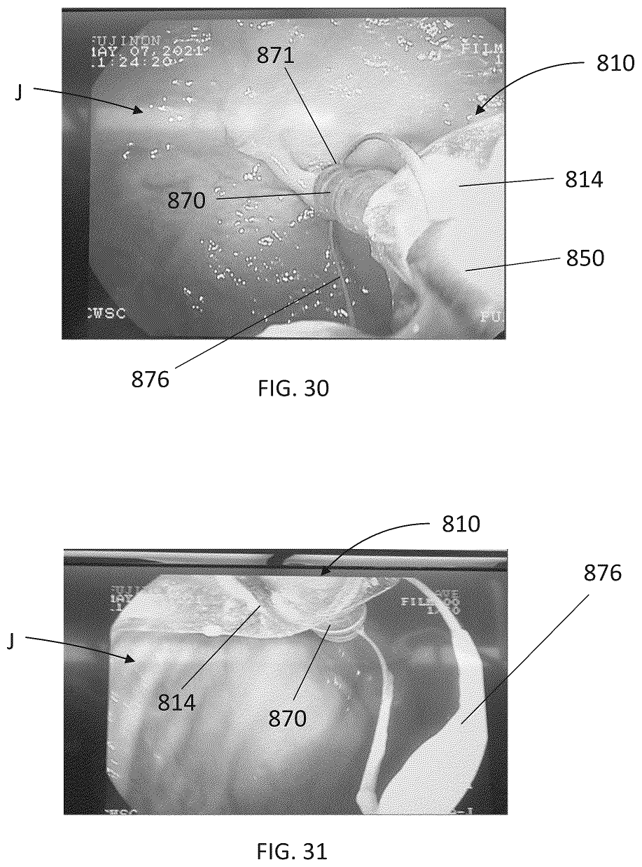

FIGS. 30 and 31 are images obtained by an endoscope 850 in an interior of a jejunum J of a patient. FIG. 30 shows the endoscope 850 coupled to an engagement portion 870 of an inflation assembly 810 via a flexible loop element 876. The inflation assembly 810 can be the same or similar in structure and/or function to any of the inflation assemblies described herein. For example, the inflation assembly 810 can include an inflatable member 814. The flexible loop element 876 is partially disposed within the opening 871 such that the endoscope 850 can be coupled to (e.g., grasp) and drag the engagement portion 870 to move the inflatable member 814 within the jejunum.

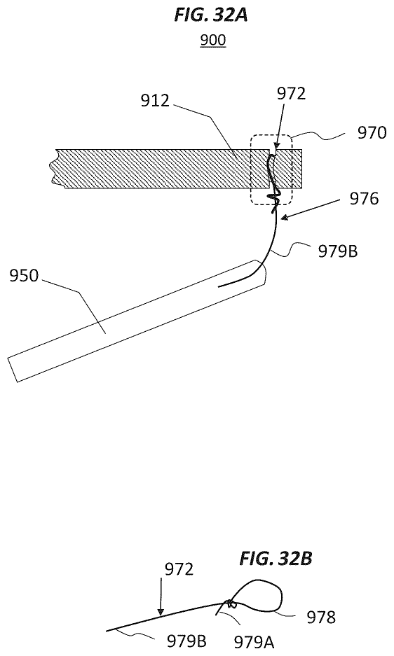

FIGS. 32A and 32B are schematic illustrations of various portions of a system 900. The system 900 can be the same or similar in structure and/or function to any of the systems described herein, such as the system 100 described above. For example, the system 900 can include an engagement portion 970 disposed at the end of an elongated tube 912 and an endoscope 950. In some embodiments the engagement portion 970 can be monolithically formed with the elongated tube 912. In some embodiments, the engagement portion 970 can be coupled to the elongated tube 912 (e.g., via adhesive or ultrasonic welding). In some embodiments, the engagement portion 970 can be any suitable device for coupling the endoscope 950 to the elongated tube 912 such that the endoscope 950 can translate (e.g., drag) an end of the elongated tube 912. In some embodiments, as shown in FIG. 32A, the engagement portion 970 of the elongated tube 912 includes a groove 972 configured to be snared by a flexible loop element 976, which can include a first end 979A and a second end 979B. The flexible loop element 972 can be formed from any suitable flexible material appropriate for surgical procedures (e.g., plastics such as fluoropolymers, fabrics, and the like). In some embodiments, the first end 979A can be attached to or gasped by the endoscope 950, while the second end 979A can be wrapped around as shown in FIG. 32B to form loop 978 configured to be disposed in the groove 972). In some embodiments, under control of a practitioner, the endoscope 950 can be configured to release the second end 979A and be removed from a body separately from the elongated tube 912 and inflatable member 914.

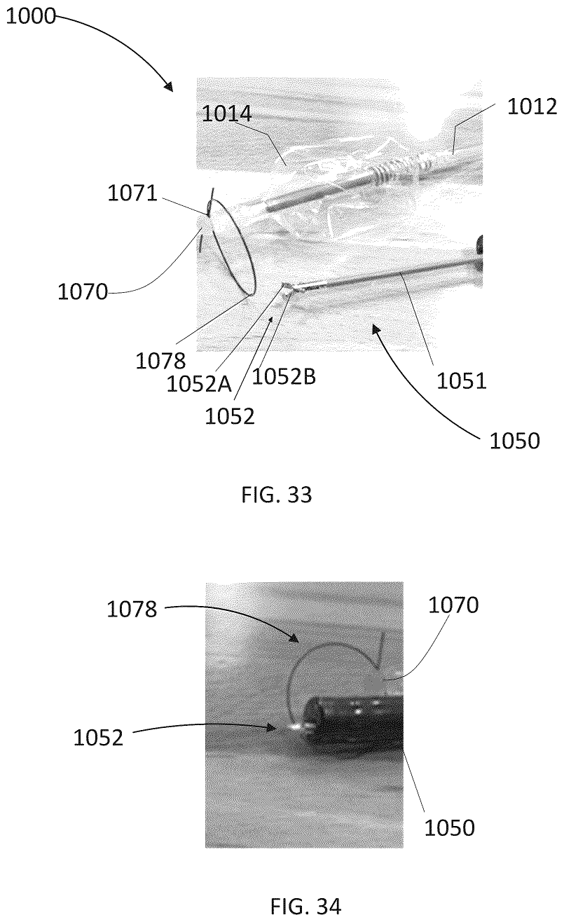

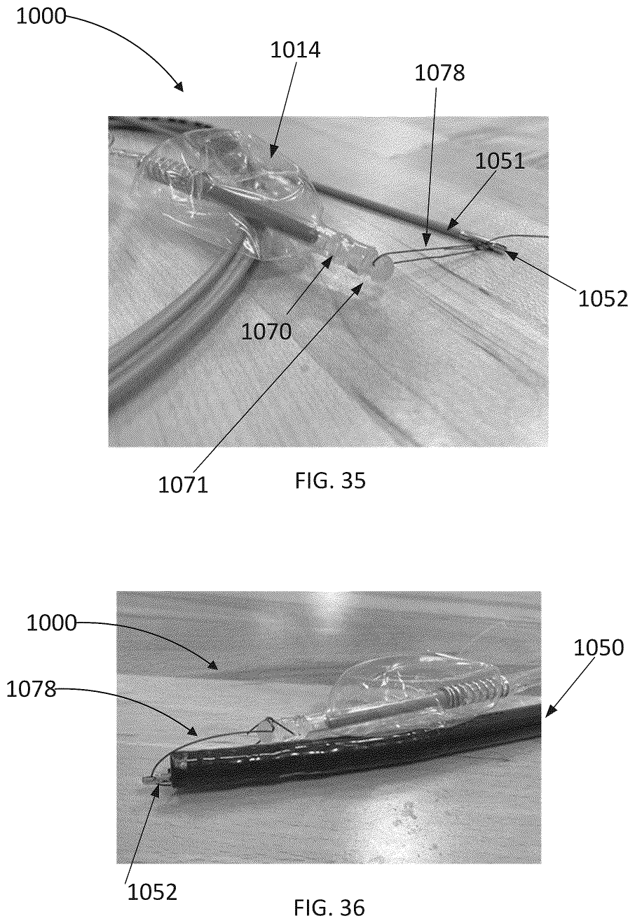

FIGS. 33-36 are various views of portions of a system 1000 in various stages of operation. FIG. 33 is a perspective view of a distal end of an inflation assembly 1010 of the system 1000 and a distal end of an endoscope 1050 of the system 1000 uncoupled from the inflation assembly 1010. FIG. 34 is a side view of an endoscope coupling mechanism 1052 of the endoscope 1050 coupled to a flexible loop 1078 of the inflation assembly 1010. FIG. 35 is a perspective view of the coupling mechanism 1052 at a distal end of an elongated grasping tube 1051 coupled to the flexible loop element 1078 of the inflation assembly 1010. FIG. 36 is a perspective view of the coupling member 1052 coupled to the flexible loop element 1078 of the inflation assembly 1010 with the elongated grasping tube 1051 disposed within an outer sheath of the endoscope 1050.

The system 1000 can be the same or similar in structure and/or function to any system described herein, such as the system 100. For example, the inflation assembly 1010 can be the same or similar in structure and/or function to any of the inflation assemblies described herein and can include an inflatable member 1014, an elongated tubular member 1012, and the engagement portion 1070. The endoscope 1050 can be the same or similar in structure and/or function to any of the endoscopes described herein. In some embodiments, the endoscope coupling mechanism 1052 can be the same as endoscope coupling mechanism 152. The flexible loop 1078 can be partially disposed within an opening 1071 defined in the engagement mechanism 1070. The coupling mechanism 1052 of the endoscope can be configured to grasp the flexible loop 1078. The coupling mechanism 1052 can be configured, for example, to transition between an open or closed configuration. For example, the coupling mechanism 1052 can include opening and closing elements 1052A and 1052B (e.g., graspers or teeth) capable of grabbing and retaining flexible element 1078 (e.g., under the control of tension cables and/or springs extending through the elongated grasping tube 1051). In FIG. 33, the elements 1052A and 1052B are in an open configuration and ready to receive a portion of the flexible loop 1078. As shown in FIG. 33, the coupling mechanism 1052 can be disposed at the end of the elongated grasping tube 1051. The elongated grasping tube 1051 can be extended and retracted relative to the outer sheath of the endoscope 1050. FIG. 34, for example, shows the endoscope in a configuration in which the elongated grasping tube 1051 is retracted into the outer sheath of the endoscope 1050.

As shown in FIGS. 35 and 36, when the coupling mechanism 1052 is closed with the flexible loop 1078 retained between the opening and closing elements 1052A and 1052B, the endoscope 1052 can be translated to drag the flexible loop 1078 and control translation of the elongated tube 1012 and the inflatable member 1014.

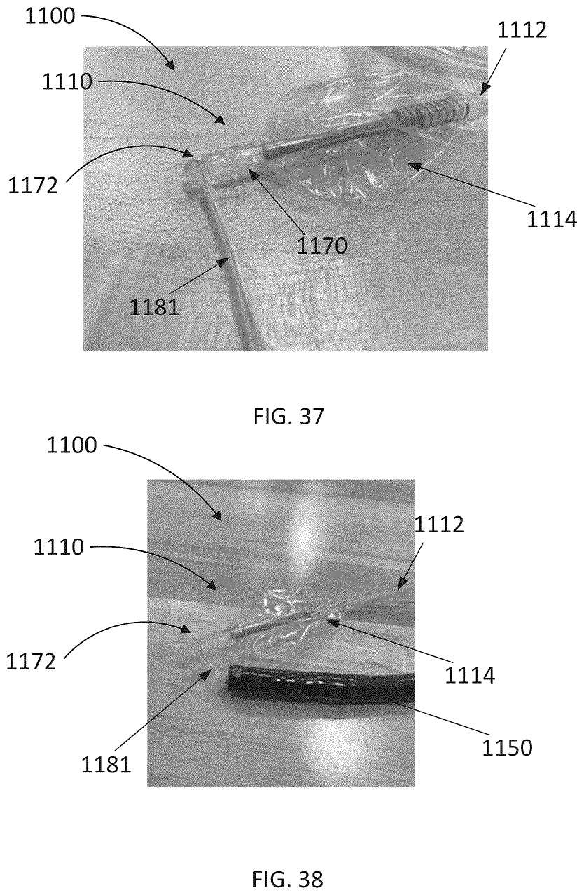

In some embodiments, rather than grasping a flexible loop of the inflation assembly with an endoscope, an endoscope can include a snare configured to be engaged with a circumferential groove of the inflation assembly. For example, FIGS. 37 and 38 depict a system 1100, which can be the same or similar in structure and/or function to any of the systems described herein. The system 1100 can include an inflation assembly 1110 and an endoscope 1150 having a flexible snare 1181. The inflation assembly 1110 can include an engagement portion 1170, an inflatable member 1114, and an elongated tube 1112. The engagement portion 1170 can be disposed on a distal end of the elongated tube 1112 and define a circumferential groove 1172 sized and shaped to receive and retain a loop portion of the flexible snare 1181. In some embodiments, the endoscope 1150 may be configured to tighten the loop portion of the flexible snare 1181 to secure the loop portion in the groove 1172. For example, the loop portion of the flexible snare 1181 can be transitioned between an open and a closed configuration.

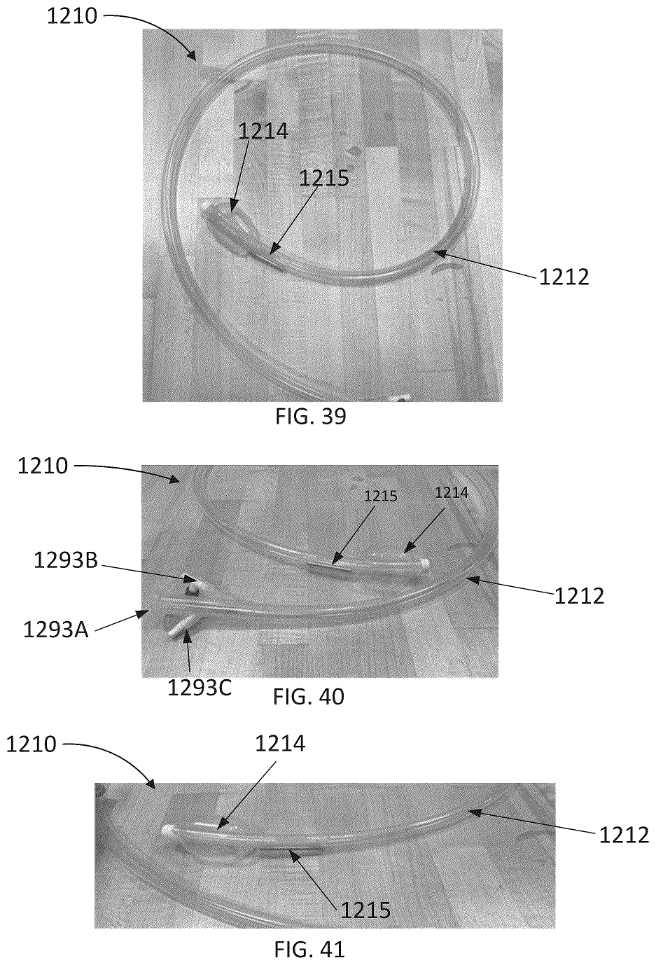

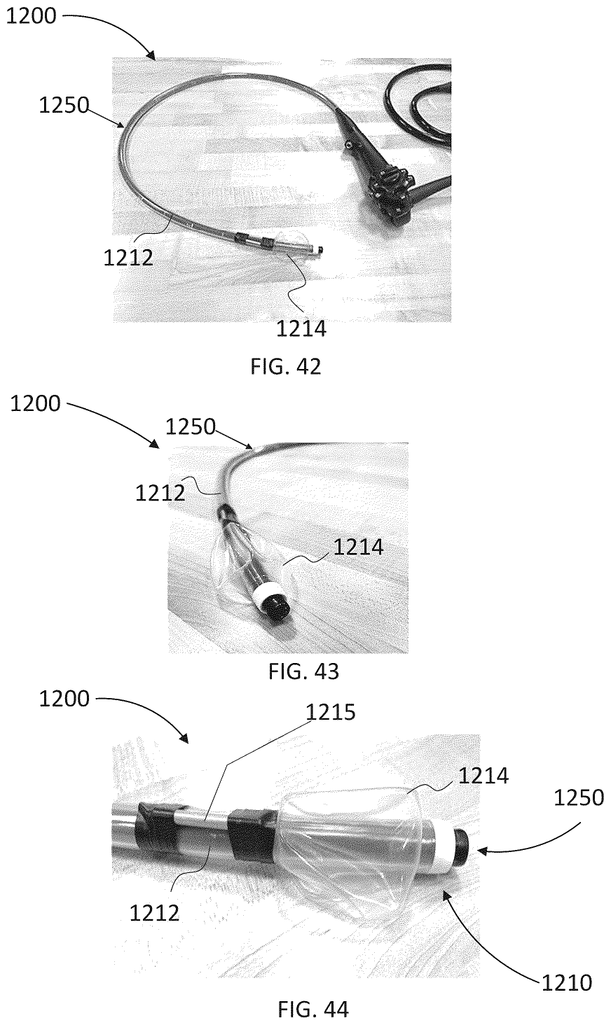

In some embodiments, rather than an inflation assembly having an engagement portion configured to be coupled to or engaged with an endoscope, the inflation assembly can define an endoscope lumen within which an endoscope can be disposed such that navigation of the endoscope through a patient's body can carry (e.g., translate) the inflation assembly 1210. For example, FIGS. 39-41 are various views of an inflation assembly 1210 of a system 1200. FIGS. 42-44 are various views of the inflation assembly 1210 with the endoscope 1250 of the system 1200 disposed within an endoscope lumen 1293 of the inflation assembly 1210. The inflation assembly 1210 can be the same or similar in structure and/or function to any of the inflation assemblies described herein. For example, the inflation assembly 1210 includes an elongated tube 1212, an inflatable member 1214, and a magnetic member 1215. The elongated tube 1212 includes an endoscope port 1293A and defines the endoscope lumen 1293 having a sufficiently large diameter to receive the endoscope 1250. In some embodiments, the inflatable member 1114 can be disposed such that the inflatable member 114 is coaxial with the elongated tube 1212 and/or the endoscope lumen 1293 and surrounds the elongated tube 1212. Although the magnetic member 1215 is shown as being coupled to an outer surface of the elongated tube 1212, in some embodiments the magnetic member 1215 can be disposed within a lumen of the elongated tube 1212 and/or within the balloon 1214. In some embodiments, the elongated tube 1212 includes ports 1293B and 1293C coupled to lumens which may be the same or similar to any of the ports or lumens described herein.

As shown in FIGS. 42-44, the endoscope 1250 can be disposed within the endoscope lumen 1293. In some embodiments, a portion of the endoscope 1250 can extend from a distal end of the inflation assembly 1210. In use, after navigating the endoscope 1250 and the inflation assembly 1210 to a desired location within a jejunum, the endoscope 1250 can be withdrawn relative to the inflation assembly 1210 such that the inflatable member 1214 can be urged toward a jejunum wall as described above.

In some embodiments, a system for performing a jejunostomy procedure includes an elongated tube having a first end and a second end, the elongated tube including an engagement portion on the first end of the elongated tube configured for engagement with an endoscope such that the endoscope can translate the first end of the elongated tube. Further the system includes an inflatable member coupled to the elongated tube, the inflatable member configured to transition from an uninflated configuration to an inflated configuration; and a magnetic member coupled to the elongated tube, the magnetic member configured such that, in response to disposing an external magnetic assembly on a surface of the abdomen of the patient, the inflatable member can be urged against an inner surface of a jejunum wall of the patient such that any intervening structure between the surface of the abdomen of the patient and the inner surface of the jejunum wall is disposed between the external magnetic assembly and the inflatable member with substantially no fluid gaps. The system can be the same or similar in structure and/or function to any of the systems described herein.

In some embodiments, an engagement portion defines an opening and includes a loop (i.e., a flexible element) partially disposed within the opening and configured to be snared by an endoscope such that the endoscope can translate the first end of the elongated tube. In some embodiments, the endoscope can snare the loop via a grasping end. Alternatively, the endoscope can snare the loop using any other suitable approach (e.g., by attaching or wrapping another flexible element to the loop).

In some embodiments, the engagement portion defines a circumferential groove such that a snare of an endoscope can couple to the engagement portion via being disposed within the circumferential groove.

In some embodiments, the magnetic member includes a plurality of discrete magnetic segments configured to move independently from adjacent magnetic segments. In some embodiments, magnetic segments may be disposed within a lumen of a flexible tube (e.g., within a lumen of the elongated tube).

In some embodiments, the system further includes a guidewire assembly including a guidewire and a coupling member, the guidewire assembly configured to be translated through an opening in an abdomen of the patient such that the coupling member engages with the inflatable member in the jejunum and the guidewire extends through the abdomen of the patient.

In some embodiments, a jejunostomy procedure may include translating a first end of an elongated tube through an orifice of a patient, through an esophagus and/or stomach of the patient, and into a jejunum of the patient such that an inflatable member and a magnetic member of the elongated tube are disposed in the jejunum of the patient, disposing an external magnetic assembly on a surface of the patient such that the magnetic member of the elongated tube is urged toward the surface of the patient and the inflatable member is disposed against an inner surface of a jejunum wall of the jejunum, and inflating the inflatable member via a lumen of the elongated tube such that the inflatable member transitions from an uninflated configuration to an inflated configuration.

In some embodiments, the procedure may include translating a coupling member of a guidewire assembly through a jejunum wall of the jejunum, the guidewire assembly including a guidewire having a first end coupled to the coupling member and a second end disposed outside the patient, the guidewire extending through the jejunum wall, and coupling the coupling member to the inflatable member.

In some embodiments, the procedure includes withdrawing the elongated tube through the orifice such that the coupling member and the first end of the guidewire are withdrawn from the orifice and such that the guidewire extends through the esophagus, stomach, jejunum and jejunum wall of the patient and the second end of the guidewire is disposed outside of the patient.

In some embodiments, the procedure may also include visualizing the location of the inflatable member via ultrasound, as well as visualizing other steps of the procedure (e.g., visualizing placement of a needle through a wall of jejunum).

In some embodiments, inflating the inflatable member includes providing a fluid including a contrast medium to the inflatable member, and further comprising visualizing the location of the inflatable member via fluoroscopy. In some embodiments, the fluid can include saline, water, dyed fluids, and the like.

In some embodiments, the procedure may also include inserting a first end of a needle through the jejunum wall of the jejunum and through a sidewall of the inflatable member, the translating of the coupling member of the guidewire assembly being at least partially through a lumen of the needle.

In some embodiments, the procedure may also include after withdrawing the elongated tube through the orifice such that the coupling member and the first end of the guidewire are withdrawn from the orifice, decoupling the coupling member and the inflatable member. In an example embodiment, an orifice of the patient is a nasal orifice or the oral orifice.

In some embodiments, the procedure may also include inserting a T-fastener through an opening in the surface of the patient such that the T-fastener retains the jejunum wall relative to the surface of the patient.

Although the systems and methods described herein are described in relation to disposing a feeding or jejunostomy tube in a patient, the systems and methods can be used for any suitable procedure. For example, the systems described herein can be used for other procedures in which the creation of an access opening is needed. In some embodiments, a method can include translating an inflatable member of an elongated tube through an orifice of a patient and to a first location within the patient. The inflatable member can be the same or similar in structure and/or function to any of the inflatable members described herein. Fluid can then be provided to the inflatable member via a lumen of the elongated tube such that the inflatable member transitions from an uninflated configuration to an inflated configuration. A coupling member of a guidewire assembly, such as any of the guidewire assemblies described herein, can be translated through a tissue wall of the patient to a second location within the patient near the first location. The guidewire assembly can include a guidewire having a first end coupled to the coupling member and a second end disposed outside the patient. The guidewire can extend through the tissue wall when the coupling member is disposed in the second location. The coupling member can then be coupled to the inflatable member. The elongated tube can then be withdrawn through the orifice such that the coupling member and the first end of the guidewire are withdrawn from the orifice and such that the guidewire extends through the orifice, the first location, and the tissue wall of the patient and the second end of the guidewire is disposed outside of the patient.

In some embodiments, a kit can include any suitable combination of components of systems described herein. For example, a kit can include an inflation assembly and a guidewire assembly. The inflation assembly can include an elongated tube, an inflatable member, and a magnetic member that can be the same or similar in structure and/or function to any of the elongated tubes, inflatable members, and magnetic members described herein. For example, the elongated tube can include a first end and a second end. The elongated tube can be configured for engagement with an endoscope such that the endoscope can translate the first end of the elongated tube. The inflatable member can be coupled to the elongated tube and can be configured to transition from an uninflated configuration to an inflated configuration. The magnetic member can be coupled to the elongated tube, and can be configured such that, in response to disposing an external magnetic assembly on a surface of the abdomen of the patient, the inflatable member can be urged against an inner surface of a jejunum wall of the patient to urge the jejunum wall toward the surface of the abdomen such that any intervening structure between the surface of the abdomen of the patient and the inner surface of the jejunum wall is disposed between the external magnetic assembly and the inflatable member with substantially no fluid gaps. The guidewire assembly can include a guidewire and a coupling member. The guidewire assembly can be configured to be translated through an opening in an abdomen of the patient such that the coupling member engages with the inflatable member in the jejunum and the guidewire extends through the abdomen of the patient. Optionally, in some embodiments, the kit can include an inflation fluid (e.g., disposed in a container such as a syringe or a vial) including a dye having a color that is atypical of bodily fluid. For example, the kit can include a syringe configured to fluidically couple to the elongated member such that the inflation fluid can be dispensed into an inflation lumen of the elongated member to inflate the inflatable member. In some embodiments, the kit can optionally include a needle configured to pierce the inflatable member and through which a portion of the guidewire assembly can be translated to engage with the inflatable member. The needle can be the same or similar in structure and/or function to any of the needles described herein. In some embodiments, the kit can optionally include a feeding tube and/or a T-fastener, which can each be the same or similar in structure and/or function to any of the feeding tubes and/or T-fasteners, respectively, described herein.

While various embodiments have been described above, it should be understood that they have been presented by way of example only, and not limitation. Where methods described above indicate certain events occurring in certain order, the ordering of certain events may be modified. Additionally, certain of the events may be performed concurrently in a parallel process when possible, as well as performed sequentially as described above.

Where schematics and/or embodiments described above indicate certain components arranged in certain orientations or positions, the arrangement of components may be modified. While the embodiments have been particularly shown and described, it will be understood that various changes in form and details may be made. Any portion of the apparatus and/or methods described herein may be combined in any combination, except mutually exclusive combinations. The embodiments described herein can include various combinations and/or sub-combinations of the functions, components, and/or features of the different embodiments described.

1. A system, comprising:
an elongated tube having a first end and a second end, the elongated tube including an engagement portion on the first end of the elongated tube configured for engagement with an endoscope such that the endoscope can translate the first end of the elongated tube;
an inflatable member coupled to the elongated tube, the inflatable member configured to transition from an uninflated configuration to an inflated configuration; and
a magnetic member coupled to the elongated tube, the magnetic member configured such that, in response to disposing an external magnetic assembly on a surface of the abdomen of the patient, the inflatable member can be urged against an inner surface of a jejunum wall of the patient to urge the jejunum wall toward the surface of the abdomen such that any intervening structure between the surface of the abdomen of the patient and the inner surface of the jejunum wall is disposed between the external magnetic assembly and the inflatable member with substantially no fluid gaps.

2. The system of claim 1, wherein the engagement portion defines an opening therethrough that is non-coaxial with a central axis of the first end of the elongated tube.

3. The system of claim 1, wherein the engagement portion defines an opening and includes a loop partially disposed within the opening and configured to be snared by an endoscope such that the endoscope can translate the first end of the elongated tube.

4. The system of claim 1, wherein the engagement portion defines a circumferential groove such that a snare of an endoscope can couple to the engagement portion via being disposed within the circumferential groove.

5.-6. (canceled)

7. The system of claim 1, wherein the engagement portion is adjacent to the inflatable member.

8. The system of claim 1, wherein the magnetic member is flexible.

9. The system of claim 1, wherein the magnetic member includes a plurality of discrete magnetic segments configured to move independently from adjacent magnetic segments.

10.-12. (canceled)

13. The system of claim 1, wherein a length of the magnetic member is substantially similar to a length of the inflatable member.

14.-15. (canceled)

16. A method, comprising:
translating a first end of an elongated tube through an orifice of a patient, through at least one of an esophagus or stomach of the patient, and into a jejunum of the patient such that an inflatable member and a magnetic member of the elongated tube are disposed in the jejunum of the patient,
disposing an external magnetic assembly on a surface of the patient such that the magnetic member of the elongated tube is urged toward the surface of the patient and the inflatable member is disposed against an inner surface of a jejunum wall of the jejunum; and
inflating the inflatable member via a lumen of the elongated tube such that the inflatable member transitions from an uninflated configuration to an inflated configuration.

17. The method of claim 16, further comprising:
translating a coupling member of a guidewire assembly through a jejunum wall of the jejunum, the guidewire assembly including a guidewire having a first end coupled to the coupling member and a second end disposed outside the patient, the guidewire extending through the jejunum wall; and
coupling the coupling member to the inflatable member.

18. The method of claim 17, further comprising:
withdrawing the elongated tube through the orifice such that the coupling member and the first end of the guidewire are withdrawn from the orifice and such that the guidewire extends through the stomach, jejunum, and jejunum wall of the patient and the second end of the guidewire is disposed outside of the patient.

19.-20. (canceled)

21. The method of claim 17, further comprising inserting a first end of a needle through the jejunum wall of the jejunum and through a sidewall of the inflatable member, the translating of the coupling member of the guidewire assembly being at least partially through a lumen of the needle.

22.-24. (canceled)

25. The method of claim 16, further comprising inserting a T-fastener through an opening in the surface of the patient such that the T-fastener retains the jejunum wall relative to the surface of the patient.

26. A system, comprising:
an elongated tube having a first end and a second end, the elongated tube defining a lumen configured to receive an endoscope such that the endoscope can translate the first end of the elongated tube to a target location within a patient;
an inflatable member coupled to the elongated tube, the inflatable member configured to transition from an uninflated configuration to an inflated configuration; and
a magnetic member coupled to the elongated tube, the magnetic member configured such that, in response to disposing an external magnetic assembly on a surface of the abdomen of the patient, the inflatable member can be urged against an inner surface of a jejunum wall of the patient to urge the jejunum wall toward the surface of the abdomen such that any intervening structure between the surface of the abdomen of the patient and the inner surface of the jejunum wall is disposed between the external magnetic assembly and the inflatable member with substantially no fluid gaps.

27. The system of claim 26, wherein the target location is a portion of the jejunum of the patient.

28. The system of claim 26, wherein the magnetic member is flexible.

29. The system of claim 26, wherein the magnetic member includes a plurality of discrete magnetic segments configured to move independently from adjacent magnetic segments.

30. The system of claim 29, wherein the plurality of discrete magnetic segments are disposed serially within a lumen of the elongated tube.

31.-32. (canceled)

33. The system of claim 26, wherein a length of the magnetic member is substantially similar to a length of the inflatable member.

34. The system of claim 26, further comprising a guidewire assembly including a guidewire and a coupling member, the guidewire assembly configured to be translated through an opening in an abdomen of the patient such that the coupling member engages with the inflatable member in the jejunum and the guidewire extends through the abdomen of the patient.

35.-42. (canceled)
